(19) United States
(12) Patent Application Publication
(10) Pub. No.: US 2025/0242124 A1
(43) Pub. Date: Jul. 31, 2025

(54) PROCESS AND DEVICE FOR LOCATING A LEAK

(71) Applicant: Drägerwerk AG & Co. KGaA, Lübeck (DE)

(72) Inventors: Gerd PETER, Lübeck (DE); Frauke ENDERS, Lübeck (DE)

(21) Appl. No.: 19/038,876

(22) Filed: Jan. 28, 2025

(30) Foreign Application Priority Data
Jan. 29, 2024 (DE) 10 2024 102 379.1

(51) Int. Cl. A61M 16/08; A61M 16/00

(52) U.S. Cl. CPC A61M 16/085; A61M 16/0003; A61M 16/024; A61M 2016/0027; A61M 2016/0033; A61M 2202/0007; A61M 2202/02; A61M 2205/15; A61M 2205/3327; A61M 2205/3334; A61M 2205/3344; A61M 2205/3368; A61M 2205/50

(57) ABSTRACT

A monitoring process and device establishes a patient fluid guide unit between a medical device and a patient-side coupling unit. A gas sample is branched off from the patient fluid guide unit at a branch point and directed to a sensor arrangement to measure the proportion of a component of the gas sample. A determination is made as to whether an indication of a leak has occurred between the branch point and a measuring position in the sensor arrangement. The section from the branch point to the measuring position is divided into at least two segments. For each segment a respective pneumatic resistance is given, which depends on the volume flow rate through the segment. If an indication of a leak is found, the pneumatic resistances and a pressure difference between the branch point and the measuring position are used to identify the segment in which the leak has occurred.

### CROSS REFERENCE TO RELATED APPLICATIONS

This application claims the benefit of priority under 35 U.S.C. § 119 of German Application 10 2024 102 379.1, filed Jan. 29, 2024, the entire contents of which are incorporated herein by reference.

### TECHNICAL FIELD

The invention pertains to a monitoring process and a monitoring device which can be used during medical treatment, in particular artificial ventilation (artificial respiration), of a patient. A patient fluid guide unit establishes a fluid connection between a medical device and a patient-side coupling unit. A gas mixture is guided from the medical device through the patient fluid guide unit to the patient-side coupling unit and/or in the opposite direction. Furthermore, the invention relates to a measuring process comprising such a monitoring process, a measuring system with such a monitoring device, and a treatment arrangement with such a measuring system.

### BACKGROUND

During medical treatment, a gas sample is branched-off from the patient fluid guide unit and guided to a measuring system with a sensor arrangement, whereby the sensor arrangement analyzes the gas sample. The sensor arrangement measures at least one property of the gas sample and in particular measures the proportion of at least one component of the gas sample, for example that of oxygen and/or carbon dioxide and/or that of an anesthetic.

A leak can suddenly occur in the measuring system. Ambient air can flow through this leak into the measuring system. As a rule, the ambient air has a different chemical composition than the gas sample to be analyzed. Therefore, ambient air flowing through the leak to the sensor arrangement can falsify measurements of the sensor arrangement while the patient is receiving medical treatment. This is undesirable. It is therefore desirable to be able to detect such a leak reliably and quickly.

### SUMMARY

It is an object of the invention to provide a monitoring process and a monitoring device which are capable of monitoring a measuring system for the occurrence of a leak, wherein the measuring system is capable of analyzing a gas sample from a patient fluid guide unit and wherein the monitoring process and the monitoring device are intended to provide greater reliability in monitoring the measuring system than known processes and devices.

The problem is solved by a monitoring process with monitoring process features according to the invention and by a monitoring device with monitoring device features according to the invention. Advantageous embodiments are disclosed herein. Advantageous embodiments of the monitoring process according to the invention are, where appropriate, also advantageous embodiments of the monitoring device according to the invention and vice versa.

Note: The order in which the process steps of a claim are listed does not necessarily specify a temporal order in which these steps are performed when the process is actually carried out.

The measuring system monitored according to the invention is configured to be used during medical treatment, in particular artificial ventilation, of a patient. At least temporarily during this medical treatment, a patient fluid guide unit establishes a fluid connection between a patient-side coupling unit and a medical device. The medical device is in particular a ventilator which generates and expels a breathable gas mixture for artificial ventilation. The patient-side coupling unit is configured to be positioned in and/or on a patient's body. A breathing mask and a tube are two examples of a patient-side coupling unit.

In this application, the breathable gas mixture usually contains oxygen and optionally at least one anesthetic and/or a gaseous medication. In this application, the expelled gas mixture is guided through the patient fluid guide unit to the patient-side coupling unit, whereby the patient-side coupling unit is arranged in and/or on the patient's body during the medical treatment. A ventilation circuit is optionally created.

It is also possible for a patient to inhale a breathable gas mixture from a stationary or mobile supply unit based solely on the patient's own respiratory activity. In this application, the supply unit acts as the medical device. The gas mixture flows from the supply unit through the patient fluid guide unit to the patient-side coupling unit. The medical device can also be a device that analyzes a gas mixture that a patient has exhaled.

A “fluid guide unit” is understood to be a component that is able to guide a fluid along a predetermined trajectory, whereby this trajectory is determined by the configuration and/or the arrangement of the fluid guide unit. Ideally, the fluid guide unit prevents the guided fluid from leaving the trajectory. A hose and a tube are two examples of a fluid guide unit.

With the monitoring process and the monitoring device according to the invention, such a measuring system can be monitored automatically. The monitoring process is carried out and the monitoring device can be used while the patient fluid guide unit permanently or at least temporarily establishes the fluid connection between the patient-side coupling unit and the medical device.

The invention can be used for a medical treatment of a patient as just described. Typically, the measuring system is used to determine at least one property of a gas sample, the gas sample having been or being branched-off (diverted) from the breathable gas mixture, preferably repeatedly branched-off as the gas mixture flows through the patient fluid guide unit. In particular, the or at least one desired property of the gas sample is the presence or the proportion (the concentration, the share) of a component of the gas sample, for example oxygen or carbon dioxide or an anesthetic or a medication, the respective proportion of several components or also the pressure or the temperature or the humidity of the gas sample.

A sensor arrangement of the measuring system is capable of measuring at least one property of a measuring gas mixture, preferably while the measuring gas mixture is in the sensor arrangement. The desired property of the measuring gas mixture is in particular the respective proportion of at least one component of the measuring gas mixture, but can also be, for example, the pressure or the temperature or the humidity. Optionally, the sensor arrangement can measure at least two different properties, in particular the respective proportion of two different components, of the measuring gas mixture.

The sample gas mixture comprises the branched-off gas sample to be analyzed. If there is no leak, the sample gas mixture usually coincides with the branched-off gas sample. In the event of a leak, the sample gas mixture usually consists of the branched-off gas sample and a gas mixture that flows from the environment through the leak to the sensor arrangement.

Note: The formulation is used that a sensor is capable of measuring a physical quantity, for example the proportion or concentration of a component in a gas mixture or a pressure or a temperature or a thermal conductivity of a gas mixture. This formulation means the following: The sensor directly measures the physical quantity or measures another physical quantity that correlates with the physical quantity to be measured and is therefore an indicator of the physical quantity to be measured. It is possible to search for the value that the physical quantity assumes at a certain position. The measurement position at which the or a correlating further variable is measured can deviate from this position. The measurement of the or each correlating variable provides a value for the physical variable to be measured.

A fluid connection between the sensor arrangement and a sample branch-off point or sample tapping point (hereinafter branch point) of the patient fluid guide unit is established or can be established with the aid of a sensor fluid guide unit. The measuring system is able to branch off at the branch point a gas sample from the patient fluid guide unit and thus from the breathable gas mixture and to guide it through the sensor fluid guide unit to the sensor arrangement. It is possible for the sensor fluid guide unit to pass through the sensor arrangement.

In many cases, a leak in the measuring system results in a mixture reaching the sensor arrangement wherein the mixture comprises the gas sample and ambient air or another gas mixture in the vicinity of the measuring system. In the event of a leak, this mixture of the gas sample and the ambient air/another gas mixture acts as the measuring gas mixture. The reason why a gas mixture from the environment reaches the sensor arrangement is as follows: As a rule, due to the leak at least temporarily a negative pressure relative to the environment occurs in the sensor fluid guide unit. One consequence of the leak is therefore that the sensor arrangement no longer analyzes the gas sample, but the mixture of the gas sample and the ambient air or another gas mixture in the environment. As a rule, the desired property of the gas sample differs from the corresponding property of the ambient air. Therefore, in the event of a leak, the measuring gas mixture no longer has the same desired property (more precisely: the property has a differing value) as the sampled gas. For example, the proportion of oxygen and/or the proportion of an anesthetic in the gas sample is usually different, usually higher, than the proportion of oxygen/the anesthetic in the ambient air. Therefore, in many cases the leak leads to an incorrect measurement result. This is why a leak is particularly undesirable.

A pressure sensor arrangement of the measuring system is able to measure the pressure at the branch point on the one hand and the pressure in the measuring system on the other. In many cases, the pressure measured at a different position in the patient fluid guide unit corresponds sufficiently accurately to the pressure at the branch point and can therefore be used as the desired pressure at the branch point. The pressure in the measuring system is measured at a measuring position in or at the sensor arrangement or in the sensor arrangement. This measuring position can be located in the sensor arrangement, and the sensor arrangement can guide through the sensor arrangement. The monitoring process according to the invention comprises the corresponding steps.

According to the invention, a determination (decision) is made as to whether or not an indication of a leak has occurred in the sensor fluid guide unit. Embodiments of the invention present implementations of how an indication of a leak is detected or how it is decided that there is no indication of a leak. Steps according to the invention are described below, which steps are carried out at least when an indication of a leak has been detected. The feature “indication of a leak” indicates that even the invention cannot rule out a false alarm relating to a leak in every case.

Thanks to the invention, in many cases such a leak is detected reliably and automatically. Preferably, a message is output with the result that an indication of a leak has been detected, in at least one form that can be perceived by a human. A user can then inspect the sensor fluid guide unit for a leak.

As already mentioned, the pressure in the measuring system is measured at a measuring position in or on the sensor fluid guide unit or in the sensor arrangement. In addition, the pressure is measured at the branch point, i.e. at the point at which a gas sample is branched-off from the patient fluid guide unit. According to the invention, a section of the sensor fluid guide unit is divided into a segment sequence wherein the section leads from the branch point to the measuring position. The segment sequence is known from the configuration of the measuring system, is predetermined and comprises at least two segments connected in series, preferably three segments connected in series.

For each segment of the segment sequence a respective pneumatic resistance—or more precisely: a respective value for the pneumatic resistance—is predetermined (given). The characteristic that a value is “predetermined” means that the value is predetermined for the respective process and the respective device in a form that can be evaluated by a computer. As a rule, the value is measured in advance, i.e. determined empirically. It is also possible to specify and use a standard value. The pneumatic resistance can differ from segment to segment.

When a gas or gas mixture flows through the segment, a pressure drop in the segment occurs due to the pneumatic resistance. The pressure drop is the product of the pneumatic resistance of the segment and the volume flow rate (volume flow, volume stream) through the segment. The volume flow rate is the volume per unit of time that flows through the segment. The greater the volume flow rate is, the greater is the pressure drop. As a rule, the pneumatic resistance depends on the volume flow rate and is often lower the greater the volume flow rate is.

Preferably, the given respective pneumatic resistance of a segment is empirically determined in advance. As a rule, it is reasonable to assume that the pneumatic resistance of a segment does not significantly depend on the chemical composition or the temperature or humidity of the gas mixture flowing through the segment.

According to the invention, the event that an indication of a leak has been detected triggers a sequence of steps. This sequence identifies the segment or at least one segment of the segment sequence that caused the indication of the leak. The steps of this triggered sequence are explained below.

The total volume flow rate is measured or captured (acquired/recorded/detected). The total volume flow rate is the volume flow rate at which the sample gas mixture flows through the sensor arrangement. The total volume flow rate is the sum of
the volume flow rate at which the gas sample is branched-off (tapped, derived) from the patient fluid guide unit at the branch point, and
the volume flow rate at which a gas from the environment flows through the leak (the leak volume flow rate).

The total volume flow rate is therefore the volume flow rate of the sample gas mixture. If there is no leak, the leak volume flow rate is zero and the gas sample is usually branched-off with the entire volume flow rate.

The actual pressure difference is determined. This pressure difference is the difference between the pressure at the measuring position and the pressure at the branch point. As mentioned above, the pressure sensor arrangement is able to measure these two pressures. In one embodiment, the pressure sensor arrangement is able to measure the pressure difference without necessarily measuring the individual pressures.

An indicator for a relative leakage volume flow rate is determined. The relative leakage volume flow rate is the ratio between
the volume flow rate which flows through a leak to be detected and which is caused by the leak, and
the total volume flow rate to the measuring position, i.e. the volume flow rate of the measuring gas mixture, whereby the total volume flow rate has been measured or captured.

The relative leakage volume flow rate is therefore a percentage (proportion) between 0% and 100%. If there is a leak, the relative leak volume flow rate is greater than 0%. The indicator determined for the relative leakage volume flow rate can be used to determine whether there is an indication of a leak or not. It is also possible that the indicator for the relative leakage volume flow rate is only determined in response to the fact that an indication of a leak has been detected in another way. Several embodiments of the inventions present different ways of determining the relative leakage volume flow rate.

The section from the branch point to the measuring position is divided into at least two segments connected in series. If this section is divided into n segments, n>=2, n−1 transitions (junctions) occur between every two neighboring segments. For the or each transition a respective transition pressure difference is calculated. The transition pressure difference is the pressure difference that would ideally occur if the leak were to occur at this transition. The term “ideal” indicates that a calculation result usually does not fully correspond to reality. To calculate a transition pressure difference, the total volume flow rate through the sensor arrangement and the relative leak volume flow rate are used on the one hand. On the other hand, the given pneumatic resistances of the segments are used.

A segment is identified which has caused the detected indication of a leak. To identify the segment, the determined actual pressure difference is used on the one hand. On the other hand, the or each calculated transition pressure difference is used.

The invention utilizes the fact that the gas sample flows on its way from the branch point to the measuring position through each segment of the segment sequence. Therefore, the gas sample is subject to the respective pneumatic resistance of each segment. In other words, the branched-off gas sample is subject to the total pneumatic resistance of the segment sequence. The total pneumatic resistance is the sum of the pneumatic resistances of the segments connected in series.

The ambient air or other gas mixture that is drawn (sucked) through the leak, on the other hand, only flows through the or each segment that is located between the leak and the measuring position on or in the sensor fluid guide unit. This leak gas mixture is therefore subject to a lower pneumatic resistance than the branched gas sample-unless the leak occurs at the branch point. Therefore, the closer the leak is positioned to the measuring position, the lower is the determined pressure difference, all other things being equal. The closer the leak is to the measuring position, the shorter the distance over which the gas mixture has to travel from the leak to the measuring position. As mentioned above, the pressure in the measuring system is measured at the measuring position on or in the sensor fluid guide unit. The fact that the pressure difference decreases the closer the leak is to the measuring position is used to identify the segment based on the actual pressure difference.

If such a leak is detected, it must be eliminated quickly. Otherwise, the patient could not receive a fully correct medical treatment. The invention not only detects the presence of a leak, but also provides information about where this leak has occurred. Thanks to the invention, in many cases it is not necessary to inspect the entire measuring system for a leak. Rather, it is often sufficient to examine the identified segment and in particular a transition of the identified segment to a neighboring segment for a leak. This advantage is often achieved because a leak often occurs at the transition between two neighboring segments. In other words: The segments can often be determined such that a leak occurs at the transition between two neighboring segments. In many cases, this effect of the invention reduces the time required to eliminate the leak. The invention therefore often reduces the overall downtime required to find and eliminate the leak. The invention therefore increases the reliability of the monitoring of the measuring system and thus the reliability of the measuring system itself.

The invention does not require a sensor between the branch point and a pressure sensor in the sensor arrangement. Rather, according to the invention, the segment is determined based on the pressure difference.

In many cases, the invention can be integrated into an existing measuring system with relatively little effort. As a rule, the invention requires
a first pressure sensor that measures the pressure at the measuring position in the sensor arrangement, and
a second pressure sensor that measures the pressure in the patient fluid guide unit.

As a rule, the pressure at the branch point matches with sufficient accuracy the pressure measured by the second pressure sensor. Two such pressure sensors are often already present, or at least the second pressure sensor. An additional pressure sensor or other sensor is generally not required to implement the invention in an existing measuring system. Therefore, the invention can often be implemented in an existing treatment arrangement by adapting a signal-processing control unit accordingly, in particular by installing and activating corresponding software and then executing this software in the control unit. This treatment arrangement is configured for a medical treatment of a patient.

According to the invention, the section from the branch point to the measuring position is divided into at least two segments. In one embodiment, the measuring system comprises a water trap located between the sensor fluid guide unit and the sensor arrangement, preferably at an inlet of the sensor arrangement. In particular, condensation and other liquids that are or may be present in the branched-off gas sample and/or in the measuring gas mixture are collected in this water trap. During artificial ventilation in particular, the breathable gas mixture flowing to the patient-side coupling unit and therefore also the gas sample have a high humidity in order not to dry out the patient's respiratory system, and a higher temperature than the ambient temperature. Therefore, a liquid often condenses on an inner surface of a fluid guide unit.

In the implementation with the water trap, the section is preferably divided into three segments. The first segment extends from the branch point to the entrance of the water trap and includes the sensor fluid guide unit. The second segment comprises the water trap, the third segment the way from the entrance of the sensor arrangement to the measuring position, which way is located in the sensor arrangement according to this implementation.

In one embodiment, the measuring system comprises its own fluid conveying unit (fluid delivering unit). A “fluid conveying unit” is understood to be a component that is capable of conveying a fluid, for example by generating a vacuum or an overpressure. A pump, a blower, a fan, and a piston-cylinder unit are examples of a fluid conveying unit. The fluid conveying unit of the measuring system can be switched on and off by an external control. The switched-on fluid conveying unit is able to branch-off a gas sample to be analyzed from the patient fluid guide unit and deliver (convey) it to the sensor arrangement. In one embodiment, the sensor arrangement is located between the branch point and the fluid conveying unit. The fluid conveying unit draws (sucks) the gas sample through the sensor arrangement.

According to the invention, the pressure in the measuring system is measured. In one embodiment, the measuring position at which this pressure is measured is located between the branch point and the fluid conveying unit of the measuring system. Preferably, a segment of the segment sequence into which the section from the branch point to the measuring position is divided is located in the sensor arrangement and upstream of the fluid conveying unit of the measuring system. The term “upstream” refers to the flow direction of the measuring gas mixture from the branch point to the sensor arrangement.

The sensor fluid guide unit preferably connects the branch point with the fluid feed unit of the measuring system. When the fluid guide unit is switched off, in many cases the gas sample flows through the sensor fluid guide unit to the sensor arrangement due to an overpressure in the patient fluid guide unit.

According to the invention, the pressure in the measuring system is measured. In a preferred embodiment, the fluid conveying unit of the measuring system is switched off while the pressure in the measuring system is measured. Preferably, the fluid conveying unit is switched on during regular operation of the measuring system and is temporarily switched off to check for a leak or at least when an indication of a leak has already been detected. Because, according to this embodiment, the pressure in the measuring system is measured while the fluid conveying unit is switched off, a negative pressure or even positive pressure or a pressure fluctuation (pressure oscillation) generated by the fluid conveying unit does not affect the pressure measurement. Instead, an equilibrium is established between the pressure at the branch point and the pressure in the sensor arrangement, ideally established at the speed of sound.

According to the invention, the total (entire) volume flow rate is measured or captured, i.e. the volume flow rate at which the measuring gas mixture flows through the sensor arrangement. In one embodiment, the measuring system comprises a separate sensor that measures the volume flow rate through the sensor arrangement. In another embodiment, the total volume flow rate is derived and measured as a result. For example, a sensor measures the volume flow rate through the patient fluid guide unit. The total volume flow rate through the sensor arrangement is derived using this volume flow rate, a cross-sectional area of the patient fluid guide unit, and a cross-sectional area of the sensor fluid guide unit.

During operation of the measuring system, the sensor arrangement measures at least one property of the measuring gas mixture that reaches the sensor arrangement. In one embodiment, the or at least one property is the proportion (concentration share) of a component of the measuring gas mixture. The component is, for example, O₂ and/or CO₂ and/or an anesthetic or a medication. If, for example, a patient's own respiratory activity is supported or even replaced by the medical device, the proportion of O₂ in the measuring gas mixture is greater during an inspiratory phase than during an expiratory phase. Conversely, the proportion of CO₂ is greater during an expiratory phase than during an inspiratory phase.

According to the invention, a determination is made as to whether an indication of a leak has occurred or not. In one embodiment, this determination is made depending on the respective time course of the respective proportion of at least two different components (constituents) of the measuring gas mixture. These two proportions serve as two measured properties of the measuring gas mixture. The two time courses are compared with each other. This embodiment utilizes the fact that the proportion of at least one component of the breathable gas mixture flowing through the patient fluid guide unit is different from the proportion of this component in the environment of the measuring system. In a leak-free state, the two time sequences are ideally out of phase (phase-shifted). The two components are, for example, oxygen and carbon dioxide.

According to the invention, an indicator of the relative leakage volume flow rate is determined. Different embodiments are possible as to how this indicator is determined.

In one embodiment, the proportion of at least one component of the measuring gas mixture is used as the or one measured property. The or each component is measured by the sensor arrangement and is, for example, O₂ and/or CO₂. As already mentioned, the measuring gas mixture is equal to the gas sample if there is no leak. The proportion of this component in the gas sample that is branched off at the branch point is also measured, for example in the patient fluid connection or by the sensor arrangement. If the proportion of the component is measured by the sensor arrangement, this measurement is preferably carried out at a time when there is no leak and therefore the gas sample reaches the sensor arrangement unaltered. The proportion of this component in the environment of the measuring system, for example in air, is known and is given in a computer-executable form. It is also possible that the proportion of the component in the environment is measured in advance and is given for the monitoring process and the monitoring device. As already mentioned, the entire volume flow rate to the measuring position is also measured or captured.

By definition, the volume flow rate through the leak is the product of the required relative leak volume flow rate, i.e. a ratio, and the total volume flow. The volume flow rate through the leak contains a volume flow rate of the component with the known and specified proportion. The measured or captured total volume flow rate contains a volume flow rate of the component with the proportion measured by the sensor arrangement. With the help of these relationships, a calculation rule, which can be evaluated by a computer, can be set up in advance, which calculation rule has the relative leak volume flow rate as the only unknown, and this calculation rule can be applied when monitoring the measuring system.

In another embodiment, the sensor arrangement measures as one property the thermal conductivity of the measuring gas mixture. According to the embodiment just described with the component, the thermal conductivity of the gas sample is measured, for example in the patient fluid guide unit or in a state free of a leak. The thermal conductivity in the environment of the measuring system, for example the thermal conductivity of the surrounding air, is known and is given (specified, predetermined). Again, a calculation rule that can be evaluated by a computer can be set up in advance, and the calculation rule can be used in the check for a leak, whereby the calculation rule again contains the relative leak volume flow rate as the only unknown.

These two embodiments can be combined with each other. Ideally, the two configurations provide the same value for the relative leakage volume flow rate, but in practice they usually provide different values. On the one hand, the combination of the two configurations makes it possible to compare the results with each other and check them for plausibility. If both configurations provide a plausible result, an average or weighted average can be used as the leakage volume flow rate.

It is also possible to decide whether an indication of a leak has occurred in addition or instead depending on the determined pressure difference. If a leak has occurred, the pressure difference usually decreases. This is because in the event of a leak part of the measuring gas mixture is subject to a lower pneumatic resistance than in the event of a leak-free state. This part of the measuring gas mixture is the part that flows through the leak.

According to the invention, the determined relative leakage volume flow rate is used to identify the segment that has caused the indication of the leak. In one embodiment, the relative leak volume flow rate is determined continuously, and the determination as to whether or not there is an indication of a leak is made depending on the relative leak volume flow rate. In particular, there is an indication of a leak if at a certain time point the relative leak volume flow rate is greater than a predetermined lower threshold (limit) and/or increases faster than a predetermined change threshold.

According to the invention, for each segment a respective pneumatic resistance is given. Preferably, this pneumatic resistance is determined in advance (is specified), preferably empirically. It is possible that the pneumatic resistance of a segment changes in the course of use and therefore of time, for example due to water or another liquid settling on an inner wall of the segment. The pneumatic resistance of at least one segment is therefore preferably determined again later. It is possible to repeat the determination of the pneumatic resistances at regular intervals. The embodiment described below makes it possible to determine the pneumatic resistances again only if there is an indication that the values used so far do no longer apply, i.e. event-controlled and not time-controlled.

According to this embodiment, an expected (predicted) pressure difference is calculated at least once, preferably when no indication of a leak has been detected. Preferably, the expected pressure difference is calculated repeatedly, for example at a predetermined frequency. The expected pressure difference is the difference between the pressure at the measuring position and the pressure at the branch point. To use the expected pressure difference, the measured or determined total volume flow rate is used on the one hand and the previously used values for the pneumatic resistances of the segments on the other. Because the segments are connected in series, in the case of a leak-free state the total pneumatic resistance to which the measuring gas mixture is subjected on the way from the branch point to the measuring position is equal to the sum of the pneumatic resistances of the segments. Furthermore, according to the invention, the actual difference between the pressure at the branch point and the pressure at the measuring position is determined.

If the determined actual pressure difference deviates from the calculated expected pressure difference by more than a given tolerance, this result is an indication that at least one previously used value for a pneumatic resistance no longer applies. A message containing this result is generated. Preferably, this message is output in at least one form that can be perceived by a human. The output of this message can be used as a trigger to determine the pneumatic resistances again.

The invention further relates to a measuring system which is capable of monitoring a medical treatment of a patient, as well as a measuring process using such a measuring system. The measuring system comprises a monitoring device according to the invention. when carrying out the measuring process, the steps of the monitoring process according to the invention are carried out. The invention also relates to a treatment arrangement which is configured for medical treatment of a patient. This treatment arrangement comprises a measuring system according to the invention.

The invention is described below by means of an embodiment example. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a ventilation circuit for artificial ventilation of a patient, where a leak has occurred in the measuring system;

FIG. 2 is a schematic view showing three segments of the measuring system in which a leak can occur, four measuring positions, and the position at which a leak has occurred;

FIG. 3 is a schematic view showing the three segments of FIG. 2 with an alternative configuration of the sensor arrangement;

FIG. 4A and FIG. 4B are graphs showing two time courses of the pressure in the sensor arrangement at two different positions of a leak;

FIG. 5 is a graph showing a pressure loss in the four measuring positions in FIG. 2 and FIG. 3 as a function of the volume flow rate;

FIG. 6 is a graph showing a dependence of the respective pneumatic resistance on the volume flow rate for the three segments in FIG. 2 and FIG. 3;

FIG. 7A, FIG. 7B and FIG. 7C together show a flow chart of a process of the embodiment example, with connections between FIG. 7A and FIG. 7B indicated at 1 and connections between FIG. 7B and FIG. 7C indicated at 2.

### DESCRIPTION OF PREFERRED EMBODIMENTS

Referring to the drawings, in the embodiment examples, the invention is used for artificial ventilation of a patient. The patient is supplied with a breathable gas mixture comprising oxygen and at least one anesthetic, optionally additionally a medication (drug). The patient is therefore anesthetized or at least sedated.

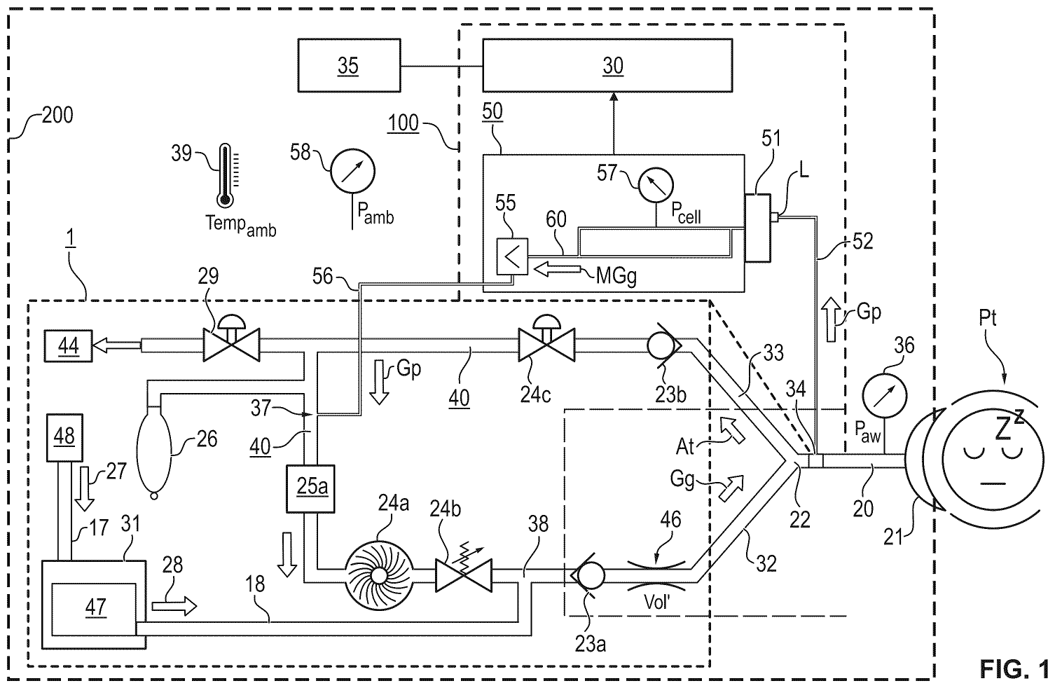

FIG. 1 schematically shows a ventilation arrangement 200 with a medical device in the form of an anesthesia device 1, whereby the ventilation arrangement 200 functions as the treatment arrangement and is able to artificially ventilate a patient Pt. Only the face of the patient Pt is shown schematically. The anesthesia device 1 maintains a gas flow in a ventilation circuit 40. The flow direction in the ventilation circuit 40 is indicated by arrows. Patient Pt is anesthetized or at least sedated.

A patient-side coupling unit 21, shown only schematically, for example a mouthpiece or a breathing mask or a tube, is arranged in and/or on the body of the patient Pt and connects the patient Pt to the ventilation circuit 40. The patient-side coupling unit 21 is connected to a Y-piece 22 by a line 20. The Y-piece 22 is connected to a gas line 32 for inhalation (inspiration) and to a gas line 33 for exhalation (expiration). The two lines 32 and 33 belong to the patient fluid guide unit of the embodiment example.

A breathable gas mixture Gg is supplied to the patient Pt in the ventilation circuit 40. In the embodiment example, this gas mixture Gg comprises oxygen (O₂), nitrogen (N₂), at least one anesthetic and optionally a further component of a carrier gas for an anesthetic, e.g. nitrous oxide (N₂O), and/or a gaseous medication. The invention can also be used for artificial ventilation in which the patient Pt is not anesthetized or sedated because the gas mixture Gg supplied to the patient Pt comprises oxygen, but no anesthetic agent. The invention can also be used, for example, to measure at least one vital parameter of the patient Pt with the aid of a medical device.

In order to deliver the gas mixture Gg to the patient Pt, the anesthesia device 1 performs a sequence of ventilation strokes. In each ventilation stroke, the anesthesia device 1 expels a quantity of the breathable gas mixture Gg. The expelled gas mixture Gg is fed in the ventilation circuit 40 to the patient-side coupling unit 21. The gas mixture At exhaled by the patient Pt into the patient-side coupling unit 21 contains anesthetic and carbon dioxide (CO₂) and flows back in the ventilation circuit 40 to the anesthesia device 1.

It is possible that the patient Pt is not completely anesthetized, but performs his/her own respiratory activity. Patient Pt's own respiratory activity is caused by his/her spontaneous breathing and optionally by external stimulation of his/her respiratory muscles. Ideally, in this application, the ventilation strokes of the anesthesia device 1 are precisely synchronized with the patient Pt's own respiratory activity.

An anesthetic vaporizer 31 generates a fluid flow 28 with an additive gas mixture comprising a carrier gas and vaporous anesthetic. The fluid flow 28 with the additional gas mixture is guided through a fluid guide unit 18 and fed into the ventilation circuit 40 at a feed point 38. The gas mixture Gg in the ventilation circuit 40 absorbs the additional gas mixture fed in.

The additional gas mixture and thus the fluid flow 28 are generated as follows: A fluid flow 27 with a carrier gas flows through a fluid guide unit 17 to a vaporization chamber 47 of the anesthetic vaporizer 31. The carrier gas in the fluid flow 27 comprises breathing air and/or pure oxygen (O₂) and optionally nitrous oxide (N₂O) and is generated by a mixer 48. In order to generate the additional gas mixture, the anesthetic vaporizer 31 vaporizes the anesthetic and feeds it into the fluid flow 27 in the vaporizer chamber 47. The anesthetic, which is fed in comes, for example, from a tank with liquid anesthetic or from a conditioner 44, whereby the conditioner 44 processes exhaled breathing air At and extracts the anesthetic from it. A vaporizer module of the anesthetic vaporizer 31 vaporizes the liquid anesthetic, and the gaseous anesthetic is fed into the fluid flow 27 in the vaporizer chamber 47.

At least as long as there is no excessive overpressure in the ventilation circuit 40, the air At exhaled by the patient Pt remains in the ventilation circuit 40, so that no anesthetic enters the environment. An overpressure is preferably reduced in such a way that no anesthetic enters the environment even when the overpressure is reduced, but instead enters the conditioner 44, for example.

Inevitably, the gas mixture At, which flows away from the patient Pt through the ventilation circuit 40, contains carbon dioxide (CO₂) exhaled by the patient Pt. A carbon dioxide absorber (CO₂ absorber) 25a is able to absorb carbon dioxide from the ventilation circuit 40.

A fluid conveying unit 24a generates a flow of a breathable gas mixture Gg in the ventilation circuit 40. The gas flow in the ventilation circuit 40 is kept running by the fluid conveying unit 24a and optionally by a manual ventilation bag 26, which can be operated manually. In the embodiment example, the fluid conveying unit 24a has the form of a blower or fan, which generates a constant fluid flow. The use of a blower makes it easier to synchronize the ventilation strokes with the patient's own respiratory activity Pt. It is also possible that the fluid conveying unit 24a comprises a pump, a bellows and/or a piston-cylinder unit.

In the embodiment example, the fluid conveying unit 24a continuously conveys the breathable gas mixture Gg. Preferably, the fluid conveying unit 24a is controlled with the control objective (control gain) that the pressure at the outlet of the fluid conveying unit 24a remains constant, for example constant 5 mbar. A controllable proportional valve 24b contributes to the generation of the individual ventilation strokes and determines the amplitudes and frequencies of these ventilation strokes. In each ventilation stroke, a quantity of the breathable gas mixture Gg is conveyed through the inhalation gas line 32 to the Y-piece 22 and to the patient-side coupling unit 21 and thus to the patient Pt.

Depending on its position, a PEEP valve 24c (PEEP=“positive end-expiratory pressure”) allows the fluid flow through the exhalation gas line 33 to pass or blocks it, thereby ensuring that a sufficiently high pressure is maintained in the lungs of the patient Pt even at the end of exhalation or if the ventilation circuit 40 is opened or interrupted for a short time. This reduces the risk of the patient Pt's lungs collapsing due to insufficient pressure.

A non-return valve (check valve) 23a allows a gas flow to pass through the inhalation gas line 32 towards the Y-piece 22 and blocks a gas flow in the opposite direction. A non-return valve 23b allows a gas flow to pass through the exhalation gas line 33 away from the Y-piece 22 and blocks a gas flow in the opposite direction.

A pressure relief valve 29 is able to reduce excess pressure (overpressure) in the ventilation circuit 40 by causing gas to escape from the ventilation circuit 40 if the pressure is too high. Preferably, the released gas is discharged into a transport line for anesthetic gas and reaches the conditioner 44. This prevents anesthetics from escaping into the environment even in the event of excess pressure.

A signal-processing ventilation control unit 35, shown only schematically, receives a signal from an optional pressure sensor 58, which measures the air pressure P_{amb} in the environment of the anesthesia device 1 and thus in the environment of the ventilation circuit 40, and a signal from an optional temperature sensor 39, which measures the ambient temperature. In addition, the ventilation control unit 35 receives a signal from a pressure sensor 36, which measures the current pressure in the ventilation circuit 40, for example the ventilation pressure (airway pressure, P_{aw}) applied to the patient Pt, in one embodiment as a differential pressure relative to the ambient pressure P_{amb}. Preferably, the pressure sensor 36 measures the pressure of the breathable gas mixture Gg, which is delivered to the patient-side coupling unit 21 in an inspiratory phase. The measuring position of the pressure sensor 36 is at the line 20, e.g. A volume flow rate sensor 46 measures the volume flow rate (volume flow) Vol′, i.e. the volume per unit of time, through the line 32 at a measuring point (measuring position) downstream of the feed point 38 and downstream of the non-return valve 23a. The ventilation control unit 35 receives a signal from the volume flow rate sensor 46, from the pressure sensor 36, and a respective signal from the optional sensors 58 and 39. Preferably, a volume flow rate sensor (not shown) measures the volume flow rate through the line 33.

The ventilation control unit 35 controls the fluid conveying unit 24a, the PEEP valve 24b, the anesthetic vaporizer 31 and other components of the ventilation circuit 40 in order to achieve the desired artificial ventilation and, optionally, anesthesia of the patient Pt. For this control, the ventilation control unit 35 processes signals from the sensors 36, 46, 39, 58 as well as specifications from a user and/or a higher-level control system.

In one embodiment, the ventilation control unit 35 performs closed-loop control. One control objective is that the actual time course of the volume flow rate Vol′ through the line 32 and/or the actual time course of the pressure P_{aw} in the line 32 follows a predetermined required time course. The first alternative is often referred to as volume-controlled regulation, the second alternative as pressure-controlled regulation. For this control, the ventilation control unit 35 processes signals from the sensor 46 and/or from the sensor 35.

It is often desired that the breathable gas mixture Gg, which is supplied to the patient Pt, fulfills a certain property. Frequently, the concentration (proportion, share) of a component (constituent) of the gas mixture Gg should be within a specified target range. In particular, the proportion of anesthetic should often be within a predetermined range. On the one hand, the proportion of anesthetic should be high enough to ensure that patient Pt is reliably anesthetized. On the other hand, the patient Pt's health must not be endangered by a too high proportion of anesthetic. In the following, the term “concentration” of a gas component in a gas mixture is used. A synonymous term is “proportion”.

To achieve this goal, it is necessary to measure the respective actual proportion of different components of the gas mixture Gg. At least one proportion can vary over time. As a rule, in particular the oxygen content and the CO₂ content oscillate in the gas mixture Gg. For this purpose, a sample of breathing gas (hereinafter: a gas sample Gp) is taken (branched-off, diverted) from the ventilation circuit 40 via a sampling hose (extraction hose/tube) 52, analyzed and fed back into the ventilation circuit 40 via a return hose 56.

The sampling hose 52 starts at a branch point 34, which in the embodiment example is positioned between the Y-piece 22 and the patient-side coupling unit 21. A breathable gas mixture Gg from the line 22 and an exhaled gas mixture At from the patient-side coupling unit 21 therefore enter the sampling hose 52 at times. At the branch point 34 there is optionally a valve, not shown, which in the closed position disconnects the sampling hose 52 from the ventilation circuit 40 and which can be controlled by the ventilation control unit 35. When the valve is fully open or left open, the sampling hose 52 is in an unrestricted fluid connection with the ventilation circuit 40. The return hose 56 leads to a confluence point 37 upstream of the carbon dioxide absorber 25a.

As can be seen in FIG. 1, the fluid conveying unit 24a is located between the branch point 34 and the entry point 37. Therefore, at least in an inspiratory phase, the fluid conveying unit 24a generates an overpressure in the branch point 34. This overpressure contributes to branching-off the gas sample Gp from the ventilation circuit 40.

The sampling hose 52 conducts the gas sample Gp to a sensor arrangement 50 with its own signal processing unit 30. This sensor arrangement 50 is spatially remote from the patient-side coupling unit 21 and from the lines 32 and 33 and also belongs, for example, to the anesthesia device 1 shown schematically. In FIG. 1, the sensor arrangement 50 is shown outside the anesthesia device 1 for clarity.

A fluid guide unit 60 leads through the sensor arrangement 50 and connects the sampling hose 52 to the return hose 56. The two hoses 52 and 56 and the fluid guide unit 60 are part of the sensor fluid guide unit of the embodiment example.

In the embodiment example, the sensor arrangement 50 comprises a fluid conveying unit in the form of a pump 55, which sucks the gas sample Gp out of the ventilation circuit 40 and draws it in through the sampling hose 52. In one embodiment, the pump 55 draws in the gas sample Gp at a constant flow rate of, for example, 0.2 l/min, with the volume flow rate having the unit of measurement [l/min], for example. In one embodiment, the sensors of the sensor arrangement 50 are located between the branch point 34 and the pump 55. Preferably, the pump 55 sucks the gas sample Gp through the sensors and/or past the sensors. The volume flow rate generated by the pump 55 can be constant or variable over time. The volume flow rate to the sensor arrangement 50 is only a fraction of the volume flow rate Vol′ at which the gas mixture Gg flows through the ventilation circuit 40 and which the volume flow rate sensor 46 measures.

As a rule, the gas mixture Gg flows through the ventilation circuit 40 at a temperature that is approximately equal to the patient's body temperature Pt. The branched gas sample Gp cools down in the sampling hose 52 approximately to the room temperature, causing moisture to condense. A water trap 51 is arranged at the inlet of the sensor arrangement 50, whereby moisture that has condensed in the sampling hose 52 is collected in the water trap 51. As a result, a gas sample Gp with a reduced water content reaches the sensor arrangement 50. The water trap 51 comprises a water tank, in which the condensed moisture is collected, and a receptacle, which is in fluid communication with the sampling hose 52 and in fluid communication with the sensor arrangement 50. The gas sample Gp thus flows from the sampling hose 52 through the receptacle of the water trap 51 into the sensor arrangement 50. The water tank is detachably connected to the receptacle. To empty the water tank, the water tank is detached from the holder, emptied and reconnected to the holder.

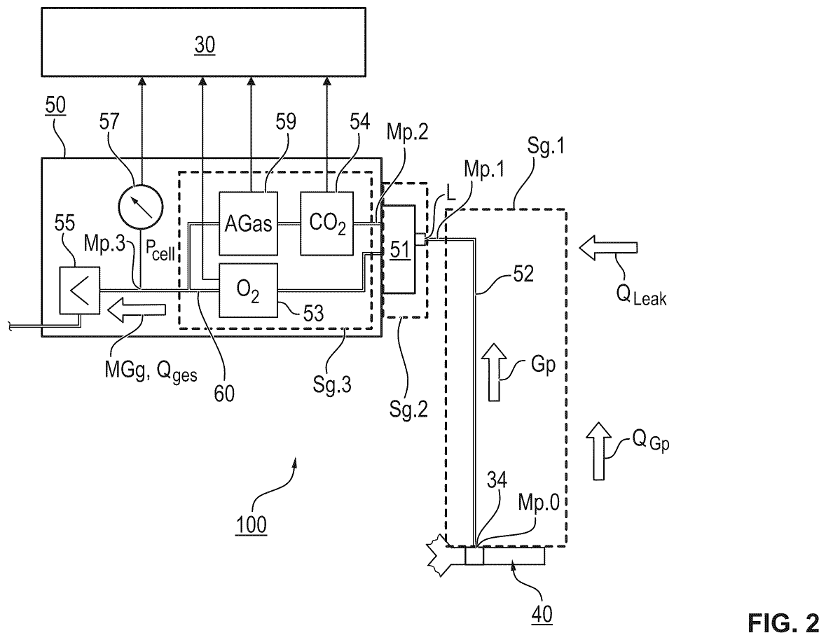

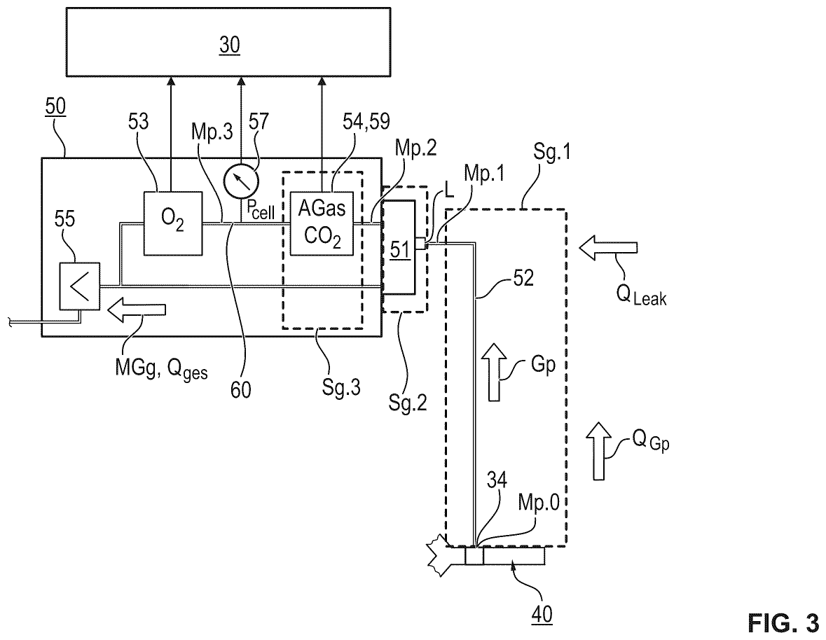

In the embodiment example, the sensor arrangement 50 comprises the following sensors, which measure the respective concentration of a component of the gas sample Gp, see FIG. 2 and FIG. 3:
a sensor 59 for the anesthetic concentration (AGas),
a sensor 54 for the carbon dioxide concentration (CO₂) and
a sensor 53 for the oxygen concentration (O₂), wherein the sensor 53 preferably utilizes the fact that oxygen is a paramagnetic gas.

The sensor arrangement 50 is configured, for example, as described in DE 10 2021 126 106 A1 (corresponding publication U.S. Pat. No. 2,023,114 548 (A1) is hereby incorporated by reference).

In addition, the sensor arrangement 50 comprises a pressure sensor 57, which measures the pressure P_{cell} of the gas sample Gp in the sensor arrangement 50. This pressure P_{cell} is variable over time because the pressure in the ventilation circuit 40 oscillates and because the sampling hose 52 is in a fluid connection with the ventilation circuit 40 if there is no valve at the branch point 34 or as long as the optional valve at the branch point 34 is open. If the valve is missing or open, the pressure in the ventilation circuit 40 propagates at approximately the speed of sound to the sensor arrangement 50.

FIG. 2 and FIG. 3 show the measuring system 100 of FIG. 1 as well as four measuring positions Mp.0, . . . , Mp.3, the meaning of which is described below. The measuring position Mp.0 is the branch point 34, the measuring position Mp.1 is the transition from the sampling hose 52 to the water trap 51, the measuring position Mp.2 is the transition from the water trap 51 to the sensor arrangement 50, and the measuring position Mp.3 is the measuring position in the sensor arrangement 50 at which the pressure sensor 57 measures the pressure P_{cell}.

In the implementation shown in FIG. 2, the two sensors 59 and 54 are connected in series and arranged on the fluid guide unit 60, and the O₂ sensor 53 is arranged parallel to these two sensors 59, 54 on a bypass line. Another arrangement is also possible. The pressure sensor 57 is arranged downstream of the three sensors 59, 54, 53 and upstream of the pump 55. The pressure sensor 57 therefore measures the pressure P_{cell} at the inlet of the pump 55.

In the implementation shown in FIG. 3, the same component implements both the sensor 59 for anesthetics and the sensor 54 for carbon dioxide. This component is connected in series with the oxygen sensor 53. This implementation reduces the risk of the three sensors 59, 54, 53 analyzing two different gas samples in the two parallel fluid guidance units. The pressure sensor 57 measures the pressure in the fluid guide unit 60, namely at a measuring position between the component that implements the sensors 54 and 59 and the O₂ sensor 53. In one embodiment, a sensor (not shown) is arranged in the bypass line, which sensor measures the density of a measuring gas mixture in the sensor arrangement 50. Other implementations and measuring positions are also possible.

Even when the pump 55 is switched off, a gas sample Gp reaches the sensor arrangement 50, unless the fluid connection between the ventilation circuit 40 and the sampling hose 52 is interrupted. In the following, the volume flow rate of the gas mixture that reaches the pressure sensor 57 is referred to as Q_{ges}. If the pump 55 is switched on, the pump 55 achieves this volume flow rate Q_{ges}. Preferably, the pump 55 is switched off for a check period, and the pressure sensor 57 measures the pressure P_{cell} during the check period.

The sensor arrangement 50, the water trap 51, the signal processing unit 30, and the hoses 52 and 56 together belong to a measuring system 100, which extracts the gas sample Gp from the ventilation circuit 40, examines it and feeds it back into the ventilation circuit 40. The measuring system 100 is part of the ventilation arrangement 200. The signal processing unit 30 receives signals from the sensors 59, 54, 53 and 57 and, in one embodiment, controls the pump 55. The measurement results of the measuring system 100 are transmitted to the ventilation control unit 35.

It is possible that a leak occurs on the path from the branch point 34 of the sampling hose 52 to the pressure sensor 57. Some possible reasons for a leak to occur are as follows:

A sensor 59, 54, 53 of the sensor arrangement 50 is replaced and the new sensor is not inserted correctly.
The sampling hose 52 is not correctly connected to the water trap 51 and/or not correctly connected to the ventilation circuit 40.
A water tank of the water trap 51 is emptied and the emptied water tank is not correctly connected to the receptacle of the water trap 51.
In order to meet hygiene requirements, the water trap 51 is replaced from time to time. However, the new water trap 51 is not correctly connected to the sampling hose 52 and/or not correctly connected to the sensor arrangement 50.
A mechanical impact causes the sampling hose 52 to become detached from the water trap 51 and/or from the ventilation circuit 40, or the sampling hose 52 is damaged.

Such a leak often occurs suddenly. FIG. 1, FIG. 2 and FIG. 3 show an example of a leak L in the transition between the sampling hose 52 and the water trap 51.

Such a leak L can falsify the measurement results of the sensors 59, 53 and/or 54 and/or of other sensors not shown. The ambient air that is drawn into the measuring system 100 through the leak L can, for example, have a higher or lower concentration of a component than the breathable gas mixture Gg. The leak L therefore fakes (simulates) a different concentration than the actual concentration and can therefore lead to an incorrect measurement result. An incorrectly measured concentration could lead to an error in the artificial ventilation of the patient Pt. Therefore, a leak L must be detected as quickly as possible, and a corresponding alarm must be issued in order to be able to localize and eliminate the leak L quickly. On the other hand, it is desirable to generate as few false alarms as possible, ideally no false alarms at all.

As already mentioned, the sensor arrangement 50 measures the respective time course of several components of a gas mixture which flows through the sensor arrangement 50. In a leak-free state, this gas mixture is equal to the branched gas sample Gp. In the following, the term “measuring gas mixture MGg” is used for the gas mixture that flows through the sensor arrangement 50. In the case of a leak L, the measuring gas mixture MGg contains a proportion of ambient air, which is drawn in through the leak L.

Various processes can be used to automatically check whether such a leak has occurred or not. In their application to the embodiment example, some processes are based on the fact that the sensor arrangement 50 measures the respective time course of at least two different components of the measuring gas mixture MGg, in particular that of CO₂ and that of O₂. The control unit 30 uses these at least two time courses to decide whether a leak has occurred or not. One basis of these processes is that, in the absence of a leak, the two curves are phase-shifted relative to each other after suitable normalization and are positioned differently relative to each other in the presence of a leak.

In one possible process, a statistical indicator of similarity between the two time courses is determined, for example a phase shift between the two time courses or an indicator of symmetry between the two time courses or a covariance between the two time courses or a covariance between the two time derivatives of the two time courses.

Another possible process involves deriving how the two time courses change over time, i.e. two time derivatives are formed. In a state free of a leak, the two time change courses ideally always have the opposite sign, because in an inspiration phase the proportion of O₂ increases and in an expiration phase the proportion of CO₂ increases. A sufficiently long period of time with the same sign is an indication of a leak.

In the embodiment example, the pump 55 is switched off in at least one monitoring period. A gas sample then flows through the sampling hose 52 into the sensor arrangement 50 due to the pressure P_{aw} in the ventilation circuit 40.

The pressure sensor 57 measures the time course of the pressure P_{cell} of the measuring gas mixture MGg at the measuring position Mp.3. The pressure sensor 36 measures the pressure P_{aw} in the ventilation circuit 40. In one embodiment of how a leak is detected, these two time courses of the pressures P_{cell} and P_{aw} are compared with each other. A large deviation is an indication of a leak.

In a further application, the sensor arrangement 50 measures the time course of the thermal conductivity of the measuring gas mixture MGg. As a rule, the breathable gas mixture Gg in the ventilation circuit 40 and therefore the gas sample Gp have a different thermal conductivity than the ambient air, so that a leak L changes the thermal conductivity of the measuring gas mixture MGg.

The signal processing unit 30 determines the time course of a relative leak volume flow rate αᵣₑₗ. The relative leak volume flow rate αᵣₑₗ is the ratio between a volume flow rate Q_{Leak} through a leak L and the total volume flow rate Q_{ges} to the sensor arrangement 50. If no leak has occurred, the relative leak volume flow rate αrel is ideally equal to zero. The total volume flow rate Q_{ges} that flows through the sensor arrangement 50 is captured or approximately measured. When the pump 55 is switched on, the total volume flow rate Q_{ges} can be captured by controlling the pump 55. When the pump 55 is switched off, the total volume flow rate Q_{ges} can be derived approximately, for example, by utilizing the fact that the volume flow rate through the line 32 at the branch point 34 is divided into a volume flow rate towards the patient-side coupling unit 21 and a volume flow rate into the sampling hose 52. The volume flow Vol′ through the line 32 is measured by the volume flow rate sensor 46. The cross-sectional areas of the line 26 and the sampling hose 52 are known from the configuration. In a leak-free state, the volume flow into the sampling hose 52 is equal to the total volume flow rate Q_{ges}. It is also possible that the measuring system 100 comprises its own volume flow sensor (not shown), which measures the total volume flow rate Q_{ges} through the sensor arrangement 30.

The total volume flow rate Q_{ges} to the measuring position Mp.3 is the sum of
the volume flow rate Q_{Gp} in the branch point 34 into the sampling hose 52 and
the volume flow rate Q_{Leak} through the leak L,
   i.e.

$${Q}_{ges} = {Q}_{Gp} + {Q}_{Leak} . \qquad ( 1 )$$

By definition

$${Q}_{L e a k} = {α}_{rel} ⋆ {Q}_{g e s} . \qquad ( 2 )$$

The following applies to the volume flow rate Q_{Gp} with which the gas sample Gp is branched-off from the ventilation circuit 34 at the branch point 34:

$${Q}_{G p} = {Q}_{ges} - {Q}_{L e a k} = ( 1 - {α}_{rel} ) ⋆ {Q}_{g e s} . \qquad ( 3 )$$

It is possible, but thanks to the invention not necessary, to measure the volume flow rate Q_{Gp} at the branch point 34.

In order to determine the relative leakage volume flow rate αᵣₑₗ, in one embodiment the signal processing unit 30 uses the measured time course of the thermal conductivity of the measuring gas mixture MGg and the given thermal conductivity of the ambient air. In another embodiment, the signal processing unit 30 additionally uses the measured time course of the concentration of O₂ or CO₂ or another component of the measuring gas mixture MGg. The two embodiments can be combined with each other.

Ideally, the thermal conductivity and the O₂ concentration of the measuring gas mixture MGg lead to the same relative leakage volume flow rate αrel, in practice these two indicators for the relative leakage volume flow rate αrel differ from each other. A suitable average of these two indicators is calculated and used as an indicator for the relative leakage volume flow rate αᵣₑₗ.

The following describes in more detail how the relative leakage volume flow rate αᵣₑₗ is determined. In many cases, it is justified to assume that the thermal conductivity WLF_{ges} of the measuring gas mixture MGg, which reaches the measuring position Mp.3, is the weighted average of the thermal conductivity WLF_{Gp} of the branched gas sample Gp and the thermal conductivity WLFₑₙᵥ of the ambient air and thus of the gas flow through the leak L. The weighting factor with which the thermal conductivity WLFₑₙᵥ of the ambient air flows in is the relative leak volume flow rate αᵣₑₗ. Under this assumption the following follows:

$$W L {F}_{g e s} = ( 1 - {α}_{rel} ) ⋆ WL {F}_{G p} + {α}_{rel} ⋆ WL {F}_{e n v} \qquad ( 4 )$$

The thermal conductivity WLFₑₙᵥ of the ambient air is known and is given (specified, predetermined). In one embodiment, the thermal conductivity WLFₑₙᵥ of the ambient air is measured regularly, for example when the sensor arrangement 50 is recalibrated. In one embodiment, a signal from the temperature sensor 39 is used to derive the thermal conductivity WLFₑₙᵥ of the ambient air. The sensor arrangement 50 measures the thermal conductivity WLF_{ges} of the measuring gas mixture MGg, which reaches the measuring position Mp.3. Preferably, the sensor arrangement 50 determines a value for the thermal conductivity WLF_{ges}, which is achieved by averaging over a breathing cycle comprising an inspiration phase, an optional intermediate phase, and an expiration phase. A different value can be determined for the next breathing cycle. If no leak has occurred, WLF_{ges}=WLF_{Gp}.

The measured thermal conductivity WLF_{ges} is used as the thermal conductivity WLF_{Gp} of the gas sample Gp, whereby the thermal conductivity WLF_{ges} used was measured at a time point or determined for a breathing cycle at which no leak was detected, for example at the start of an application or if no other process provides an indication of a leak. Under these conditions, the only unknown in equation (4) is the relative leak volume flow rate αᵣₑₗ.

The sensor arrangement 50 measures the current concentration (the current proportion/share) Conges of a component of the measuring gas mixture MGg, which is drawn in by the pump 55. Again, the assumption is justified that the concentration of this component is a weighted average of the concentration Con_{Gp} in the gas sample Gp and the concentration Conₑₙᵥ in ambient air, i.e. that

$$C o {n}_{g e s} = ( 1 - {α}_{rel} ) ⋆ Co {n}_{G p} + {α}_{rel} ⋆ Co {n}_{e n v} \qquad ( 5 )$$

Again, the concentration Conges is measured at a time when there is no leak and is used as the concentration Con_{Gp} of the gas sample Gp. The concentration Conₑₙᵥ of the component in ambient air is known or is measured in advance and is given. Equation (5) then again has the required leak volume flow rate αrel as the only unknown. It is possible that the procedure just described is carried out for two different components of the measuring gas mixture MGg, for example for O₂ and for CO₂.

At least two of the embodiments just described for detecting a leak L can be combined with each other. If, for example, at least one of the above criteria is fulfilled, there is an indication of a leak. For example, there is an indication of a leak L if the relative leak volume flow rate αrel is greater than a predetermined lower threshold.

The processes just mentioned by way of example enable a determination as to whether or not a leak has occurred between the two measuring positions Mp.0 and Mp.3 of FIG. 2—more precisely: whether an indication of such a leak has occurred. If such a leak L has occurred, it should be eliminated as quickly as possible so as not to jeopardize the artificial ventilation of the patient Pt. In order to quickly eliminate a detected leak L, a user must find the location where the leak has occurred. The invention supports the user in finding the leak L or in ruling out the possibility that a leak has actually occurred.

For this purpose, the path from the branch point 34 (measuring position Mp.0) to the measuring position Mp.3 is divided in advance into at least two segments, in the embodiment example into three segments. A user is not only shown that a leak has occurred, but also in which segment.

The at least two segments are connected in series. FIG. 2 and FIG. 3 show examples of the three segments Sg.1, Sg.2, Sg.3 of the embodiment example. The first segment Sg.1 comprises the sampling hose 52 and extends from the branch point 34 (measuring position Mp.0) to the entry into the water trap 51 (measuring position Mp.1). The second segment Sg.2 comprises the water trap 51. The leak L shown as an example has occurred in the second segment Sg.2. The third segment Sg.3 comprises the sensor arrangement 50 between the inlet (measuring position Mp.2) and the pressure sensor 57 (measuring position Mp.3).

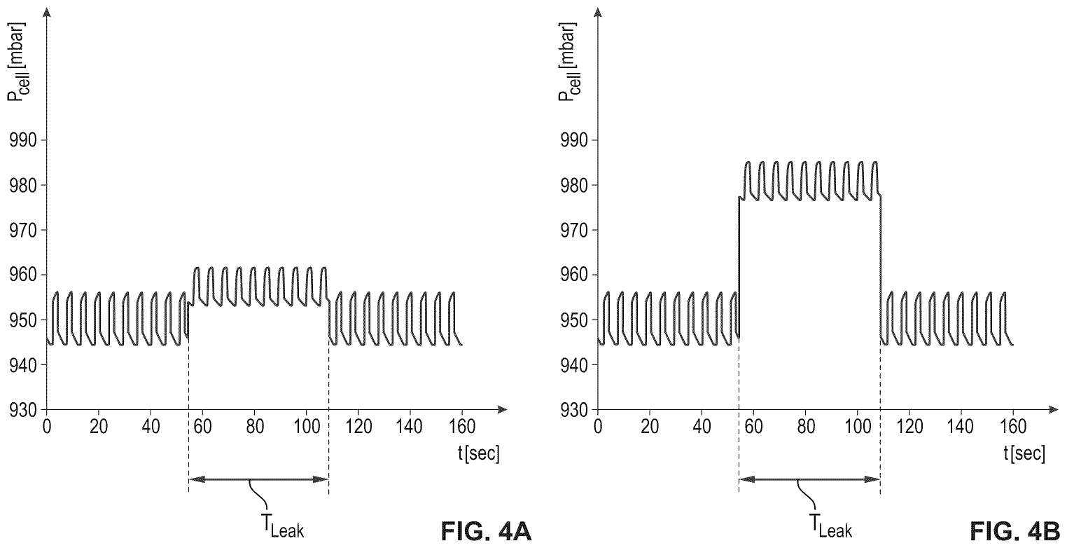

FIGS. 4A and 4B show an example for the time course of the pressure P_{cell} measured by the pressure sensor 57. The pressure P_{aw} in the ventilation circuit 40 and therefore also the measured pressure P_{cell} oscillate. The time in [sec] is plotted on the x-axis and the measured pressure P_{cell} in [mbar] is plotted on the y-axis. The effect of a leak L is visible in the time span T_{Leak} between 60 sec and 110 sec. In the example shown, an internal laboratory test was carried out in which this leak L was generated and later eliminated. The leak L causes the measured oscillating pressure P_{cell} to be greater than in a condition without a leak. In the example in FIG. 4B, the leak has occurred in segment Sg.1, in the example in FIG. 4B in segment Sg.3. It can be seen that the oscillating pressure P_{cell} is greater with a leak in segment Sg.3 than with a leak in segment Sg. 1 and greater with a leak than in a leak-free state. This fact is exploited in accordance with the invention. Why this is the case and how it is exploited to localize the leak L is explained below.

As is known, a fluid guide unit has a pneumatic resistance that causes a pressure drop. A pneumatic resistance corresponds to an electrical resistance and is also referred to as R below. According to Ohm's law, the electrical resistance is the quotient of voltage U and current I and causes a voltage drop ΔU. Correspondingly, the pneumatic resistance R is the quotient of a pressure loss ΔP and a volume flow rate Q. This pneumatic resistance R has, for example, the measuring unit mbar/(l/min), which corresponds to the electrical measuring unit Ohm. The pneumatic resistance R leads to a pressure drop ΔP. The pneumatic resistance between the measuring positions Mp.0 and Mp.3 of FIG. 2 causes a pressure drop (pressure loss) ΔP_{cell}. This pressure loss ΔP_{cell} is equal to the difference between
the pressure P_{cell} measured by the pressure sensor 57 and
the pressure P_{aw} measured by the pressure sensor 36.

The pressure loss ΔP_{cell} is measured continuously.

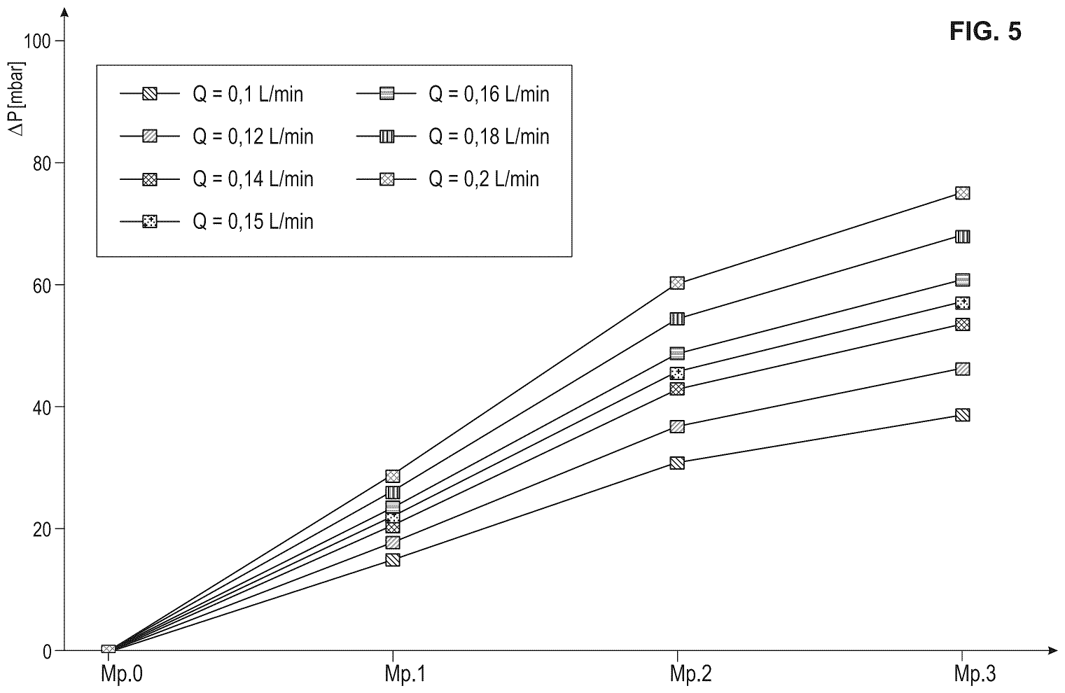

The respective pressure loss due to the pneumatic resistances of the three segments is illustrated in FIG. 5. The curves shown are from internal tests and simulations. The curves were obtained in a situation in which no leak occurred. The four measuring positions Mp.0, . . . , Mp.3, at which the respective pressure was measured, are plotted on the x-axis, and the pressure loss ΔP in [mbar], which occurs between the branch point 34 (measuring position Mp.0) and the respective measuring position Mp.0, . . . , Mp.3, is plotted on the y-axis. As already mentioned, the pressure loss ΔP is the difference between the pressure at the respective measuring position Mp.0, . . . , Mp.3 and the pressure P_{aw} at the branch point 34. The pressure loss ΔP at the measuring position Mp.3 is equal to the pressure loss ΔP_{cell} measured during use.

The pneumatic resistance R and therefore the pressure loss ΔP depend on the volume flow rate Q in [l/min] at which the gas sample Gp flows from the branch point 34 to the sensor arrangement 50. The seven measurement curves refer to volume flow rates Q between 0.1 l/min and 0.2 l/min. It can be seen that the higher is the volume flow rate Q, the greater is the pressure loss ΔP.

Note: In order to obtain the test results shown in FIG. 5, the respective pressure was measured at four different measuring positions. For productive use, it is sufficient for the pressure sensor 57 to measure the pressure P_{cell} in the sensor arrangement 50, i.e. at the measuring position Mp.3, and for the pressure sensor 36 to measure the pressure in the ventilation circuit 40 and thus at the measuring position Mp.0.

One concept according to the invention is to measure this pressure loss ΔP_{cell} and then to identify the segment Sg.1, Sg.2, Sg.3 in which the leak L has occurred, depending on the measured pressure loss ΔP_{cell}. Because the segments Sg.1, Sg.2, Sg.3 are connected in series, the pneumatic resistances of the segments add up to a total pneumatic resistance of the fluid connection between the measuring positions Mp.0 and Mp.3.

The following boundary conditions are taken into account:
The respective pneumatic resistance of a segment Sg.1, Sg.2, Sg.3 depends on the actual volume flow rate Q_{Sg.1}, Q_{Sg.2}, Q_{Sg.3} through this segment. In a state without a leak, with sufficient accuracy this volume flow rate Q_{Sg.1}, Q_{Sg.2}, Q_{Sg.3} coincides with the total volume flow rate Q_{ges} generated by the pump 55, i.e. it is the same in all three segments Sg.1, Sg.2, Sg.3. In the event of a leak L, two volume flow rates Q_{Sg.1}, Q_{Sg.2}, Q_{Sg.3} can differ from each other through the segments Sg.1, Sg.2, Sg.3. This is because the volume flow rate Q_{ges} generated by the pump 55 is then divided into the volume flow rate Q_{Gp} from the branch point 34 and the volume flow rate Q_{Leak} through the leak L.
The pressure in the ventilation circuit 40 and therefore also the pressure P_{cell}, which is measured by the pressure sensor 57, oscillates. These oscillations are caused by the ventilation strokes of the ventilator 1 and optionally by the patient's own breathing activity.
The pneumatic resistance of a segment Sg.1, Sg.2, Sg.3 can change over time, particularly due to condensation in one segment Sg.1, Sg.2.

The following describes how these boundary conditions are taken into account.

In the example, the pressure loss ΔP can be described by the following formula:

$$Δ {P}_{cell} = Δ {P}_{S g . 1} + Δ {P}_{Sg \text{.2}} + Δ {P}_{S g . 3} = {R}_{S g . 1} ( {Q}_{S g . 1} ) ⋆ {Q}_{S g . 1} + {R}_{Sg \text{.2}} ( {Q}_{S g . 2} ) ⋆ {Q}_{S g . 2} + {R}_{S g . 3} ( {Q}_{S g . 3} ) ⋆ Q {.}_{Sg \text{.3}} \qquad ( 6 )$$

Here, Q_{Sg.1}, Q_{Sg.2}, Q_{Sg.3} denote the respective actual volume flow rate through the respective segment Sg.1, Sg.2, Sg.3. ΔP_{Sg.1}, ΔP_{Sg.2}, ΔP_{Sg.3} denote the respective pressure loss in the segment Sg.1, Sg.2, Sg.3 and R_{Sg.1}(Q), R_{Sg.2}(Q), R_{Sg.3}(Q) denote the respective pneumatic resistance of the segment Sg.1, Sg.2, Sg.3. This pneumatic resistance depends on the volume flow rate Q through the segment. Formula (6) applies both to a situation without a leak and to a situation with a leak. If there is no leak, the actual volume flow rates Q_{Sg.1}, Q_{Sg.2}, Q_{Sg.3} are the same. During regular operation, they are all equal to the constant volume flow rate Q_{ges} over one that the pump 55 achieves.

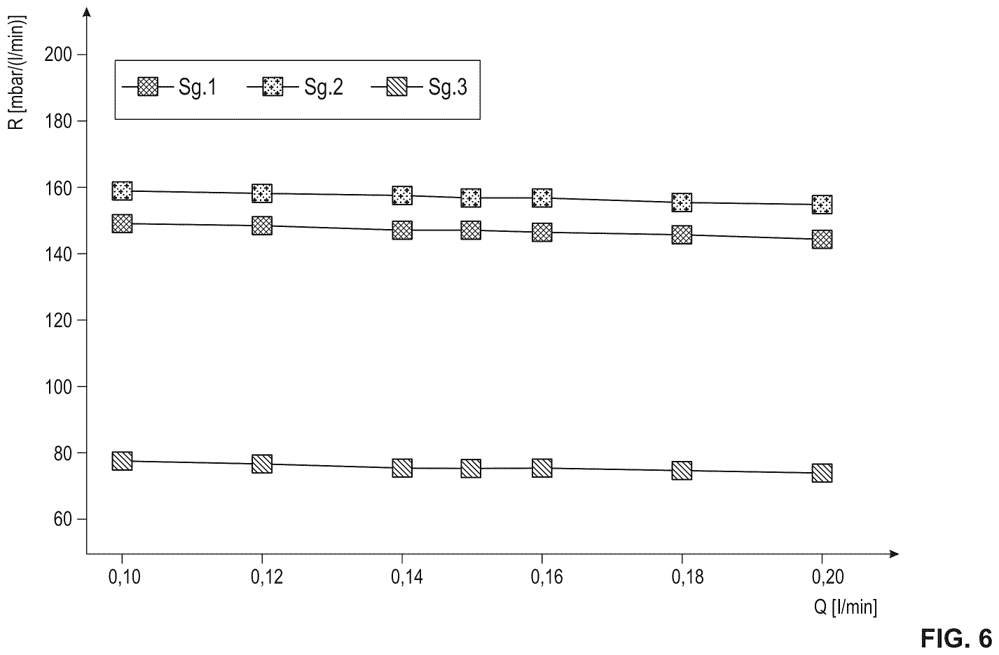

FIG. 6 shows an example of the respective pneumatic resistance R_{Sg.1}(Q), R_{Sg.2}(Q), R_{Sg.3}(Q) of the three segments Sg.1, Sg.2, Sg.3. If Ohm's law were applied directly, the pneumatic resistance R would not depend on the volume flow rate Q. In the present case, however, the pneumatic resistance R depends slightly on the volume flow rate Q, i.e. R=R(Q). The volume flow rate Q through the segment in [l/min] is plotted on the x-axis, and the pneumatic resistance R=R(Q) in [mbar/(l/min)] is plotted on the y-axis as a function of the volume flow rate Q through the respective segment Sg.x (x=1,2,3).

As can be seen in FIG. 6, the pneumatic resistance R_{Sg.x}=R_{Sg.x}(Q) of a segment Sg.x is approximated by the model

$${R}_{Sg . x} ( Q ) = R\_ {0}_{Sg . x} - R\_ 1 * {Q}_{Sg . x} \qquad ( 7 )$$

(x=1,2,3). Here, R_0_{Sg.x} and R_1_{Sg.x} are two constants greater than zero. These constants can be determined empirically in advance.

If formula (7) is inserted into formula (6), the following formula is obtained:

$$Δ {P}_{cell} = Δ {P}_{S G . 1} ( {Q}_{S g . 1} ) + Δ {P}_{Sg \text{.2}} ( {Q}_{Sg \text{.2}} ) + Δ {P}_{SG \text{.3}} ( {Q}_{Sg \text{.3}} ) = {R}_{S g . 1} ( {Q}_{S g . 1} ) ⋆ {Q}_{S g . 1} + {R}_{Sg \text{.2}} ( {Q}_{S g . 2} ) ⋆ {Q}_{Sg \text{.2}} + \text{} {R}_{S g . 3} ( {Q}_{S g . 3} ) ⋆ {Q}_{Sg \text{.3}} = \text{} [ R\_ 0 ⋆ {Q}_{S g \text{.1} Sg \text{.1}} + R\_ {0}_{Sg \text{.2}} ⋆ {Q}_{Sg \text{.2}} + R\_ {0}_{Sg \text{.3}} ⋆ {Q}_{S g . 3} ] - \text{} [ R\_ {1}_{Sg \text{.1}} ⋆ {Q}_{S g . {1}^{2}} + R\_ {1}_{Sg \text{.2}} ⋆ {Q}_{S g . {2}^{2}} + R\_ {1}_{Sg \text{.3}} ⋆ {Q}_{S g . {3}^{2}} ] . \qquad ( 8 )$$

If no leak has occurred, the formula (8) is simplified to

$$Δ P = [ R\_ {0}_{Sg \text{.1}} + R\_ {0}_{Sg \text{.2}} + R\_ {0}_{Sg \text{.3}} ] ⋆ {Q}_{g e s} - [ R\_ {1}_{Sg \text{.1}} + R\_ {1}_{Sg \text{.2}} + R\_ {1}_{Sg \text{.3}} ] ⋆ {Q}_{g e s}^{2} . \qquad ( 9 )$$

The following describes how the segment Sg.x in which this leak L has occurred (x=1,2,3) is identified after a leak L has been detected. If a leak L has occurred, the relative leak volume flow rate αrel is greater than 0. In the embodiment example, this relative leak volume flow rate αrel is determined, for example using calculation rule (4) and/or (5). In addition, the pressure difference ΔP_{cell} is determined, i.e. the difference between the two pressures P_{cell} and P_{aw} measured by the two pressure sensors 57 and 36. Although the measured pressures P_{cell} and P_{aw} oscillate, the pressure difference ΔP_{cell} oscillates less, ideally not at all.

On its way from the branch point 34 to the pressure sensor 57 the branched-off gas sample Gp is subject to the respective pneumatic resistance of all three segments Sg.1, Sg.2, Sg.3. The volume flow rate into the sampling hose 52 is Q_{Gp}. However, the gas mixture that is drawn in through the leak L and flows to the pressure sensor 57 with the volume flow rate Q_{Leak} is subject to a lower pneumatic resistance, depending on where the leak L has occurred-unless the leak has occurred at the measuring position Mp.0. The pressure difference ΔP_{cell} is the lower the closer the leak is to the pressure sensor 57 on the path from Mp.0 to Mp.3. This principle is used to identify the segment Sg.x with the leak L. This is explained below using two examples.

As already mentioned, the gas mixture from the environment flows through the leak L with a volume flow rate of Q_{Leak}=αᵣₑₗ*Q_{ges} to the pressure sensor 57, see formula (2). The relative leak volume flow rate αrel is determined, and the total volume flow rate Q_{ges} with which the measuring gas mixture MGg flows to the pressure sensor 57 is known or captured or is also measured. In particular, the volume flow rate achieved by the pump 55 is known.

The predicted pressure difference in the event that the leak L occurs at the measuring position Mp.x is denoted by ΔP_{Mp.x}. In other words: If the model assumptions described below apply and a leak L occurs at the measuring position Mp.x, but otherwise no leak occurs in the measuring system 100, and if the pressures P_{cell} and P_{aw} are measured correctly, then ΔP_{cell}=ΔP_{Mp.x}.

In the first example, it is assumed that the leak L has occurred at the measuring position Mp.2, i.e. at the transition between the segments Sg.2 and Sg.3, cf. FIG. 2, and that no further leak has occurred. At least when the pump 55 is switched on, the total volume flow rate Q_{ges}, which is generated by the pump 55 and with which the measuring gas mixture MGg reaches the pressure sensor 57, is not changed by the leak L. The gas sample Gp flows at the reduced flow rate

$${Q}_{G p} = {Q}_{g e s} - {Q}_{L e a k} = ( 1 - {α}_{rel} ) ⋆ {Q}_{g e s} \qquad ( 10 )$$

from the branch point 34 to the pressure sensor 57. The gas mixture through the leak L then flows with the volume flow rate Q_{Leak} only through the segment Sg.3 to the pressure sensor 57.

According to the calculation rule (6), the pressure loss ΔP_{Mp.2} for this gas mixture is the sum of ΔP_{Sg.1}+ΔP_{Sg.2} on the one hand and ΔP_{Sg.3} on the other. With the model assumption according to formula (7), the following applies to the pressure loss ΔP_{Sg.1}+ΔP_{Sg.2} in the two segments Sg.1 and Sg.2

$$Δ {P}_{Sg \text{.1}} + Δ {P}_{Sg \text{.2}} = [ {R}_{S g . 1} ( {Q}_{G p} ) + {R}_{S g . 2} ( {Q}_{G p} ) ] ⋆ {Q}_{G p} = [ R\_ {0}_{Sg \text{.1}} + R\_ {0}_{Sg \text{.2}} ] ⋆ ( 1 - {α}_{rel} ) ⋆ {Q}_{g e s} - [ R\_ {1}_{Sg \text{.1}} + R\_ {1}_{S g . 2} ] ⋆ {( 1 - {α}_{rel} )}^{2} ⋆ {Q}_{g e s}^{2} . \qquad ( 11 )$$

The following applies to the pressure loss ΔP_{Sg.3} in segment Sg.3

$$Δ {P}_{Sg \text{.3}} = {R}_{Sg \text{.3}} ⋆ {Q}_{g e s} = R\_ {0}_{Sg \text{.3}} ⋆ {Q}_{g e s} - R\_ {1}_{Sg \text{.3}} ⋆ {Q}_{g e s}^{2} . \qquad ( 12 )$$

The following applies to the total pressure loss ΔP_{Mp.2}

$$Δ {P}_{Mp \text{.2}} = [ R\_ {0}_{Sg \text{.1}} + R\_ {0}_{Sg \text{.2}} ] ⋆ ( 1 - {α}_{rel} ) ⋆ {Q}_{g e s} + R\_ {0}_{Sg \text{.3}} ⋆ {Q}_{g e s} - [ R\_ {1}_{Sg \text{.1}} + R\_ {1}_{Sg \text{.2}} ] ⋆ {( 1 - {α}_{rel} )}^{2} ⋆ {Q}_{g e s}^{2} - R\_ {1}_{Sg \text{.3}} ⋆ {Q}_{g e s}^{2} . \qquad ( 13 )$$

All variables on the right-hand side of formula (13) are known. So if the leak L occurs at the measuring position Mp.2, a pressure difference ΔP_{Mp.2} is measured according to the model assumption, i.e. ideally according to formula (13).

In the second example, it is assumed that the leak L has occurred at the measuring position Mp.1. This situation is shown in FIG. 2. The leak gas mixture then flows through the two segments Sg.2 and Sg.3 at the flow rate Q_{Leak} and is subject to the pneumatic resistance R_{Sg.2}+R_{Sg.3}. If no further leak has occurred, the gas sample Gp flows with the volume flow rate Q_{Gp} from the branch point 34 to the pressure sensor 57.

The pressure loss ΔP_{Mp.1} for this gas mixture is the sum of ΔP_{Sg.1} on the one hand and ΔP_{Sg.2}+ΔP_{Sg.3} on the other. Corresponding to the presentation for the volume flow rate in the event of a leak at measuring position Mp.2, the following applies to the pressure loss in Mp.1

$$Δ {P}_{Sg \text{.1}} = R\_ {0}_{Sg \text{.1}} ⋆ ( 1 - {α}_{rel} ) ⋆ {Q}_{g e s} - R\_ {1}_{Sg \text{.1}} ⋆ {( 1 - {α}_{rel} )}^{2} ⋆ {Q}_{g e s}^{2} \qquad ( 14 )$$

$$and$$

$$Δ {P}_{Sg \text{.2}} + Δ {P}_{Sg \text{.3}} = [ R\_ {0}_{Sg \text{.2}} + R\_ {0}_{Sg \text{.3}} ] ⋆ {Q}_{g e s} - [ R\_ {1}_{Sg \text{.2}} ⋆ R\_ {1}_{Sg \text{.3}} ] ⋆ {Q}_{g e s}^{2} . \qquad ( 15 )$$

The following applies to the total pressure loss ΔP_{Mp.1}

$$Δ {P}_{Mp \text{.1}} = [ R\_ {0}_{Sg \text{.1}} ⋆ ( 1 - {α}_{rel} ) ⋆ {Q}_{g e s} + [ R\_ {0}_{Sg \text{.2}} + R\_ {0}_{Sg \text{.3}} ] ⋆ {Q}_{g e s} - R\_ {1}_{Sg \text{.1}} ⋆ {( 1 - {α}_{rel} )}^{2} ⋆ {Q}_{g e s}^{2} - [ R\_ {1}_{Sg \text{.2}} + R\_ {1}_{Sg \text{.3}} ] ⋆ {Q}_{g e s}^{2} . \qquad ( 16 )$$

ΔP_{Mp.1} is greater than ΔP_{Mp.2}.

As already explained, the pressure difference ΔP_{cell} is greater the further away the leak L is from the pressure sensor 57. “Further away” refers to the path of the gas sample Gp from the branch point 34 to the pressure sensor 57. Based on this principle, the following decision rule is derived and applied during use, preferably when the pump 55 is switched on and generates the volume flow rate Q_{ges}:
If the measured pressure difference ΔP_{cell} is less than ΔP_{Mp.2}, the leak is in segment Sg.3.
If the measured pressure difference ΔP_{cell} is greater than ΔP_{Mp.1}, the leak is in segment Sg.1.
Otherwise the leak L is in segment Sg.2 or in one of the measuring positions Mp.1 or Mp.2.

The procedure just described uses the three pneumatic resistances R_{Sg.1}(Q), R_{Sg.2}(Q), R_{Sg.3}(Q) of the three segments Sg.1, Sg.2, Sg.3. In one embodiment, formula (7) is used for this, which requires two values R_0_{Sg.x} and R_1_{Sg.x} for each of the three segments Sg.x (x=1,2,3). The pneumatic resistance of a segment Sg.x can change in the course of use. If the pneumatic resistance R_{Sg.x}(Q) of a segment Sg.x changes, new values R_0_{Sg.x} and R_1_{Sg.x} are preferably determined empirically. The procedure described below describes an embodiment of how an indication is automatically found that the pneumatic resistance of at least one segment Sg.1, Sg.2, Sg.3 has changed.

According to this embodiment, it is checked in a state free of a leak whether the expected pneumatic resistance corresponds sufficiently accurately to the actual pneumatic resistance. The expected pressure difference ΔPₑₓₚ is predicted according to formula (6), whereby the previously used values for the pneumatic resistances R_{Sg.1}(Q), R_{Sg.2}(Q), R_{Sg.3}(Q) of the three segments Sg.1, Sg.2, Sg.3 are used in formula (6). As no leak occurs, the volume flow rates through the segments are equal to Q_{ges}. The actual pressure difference ΔP_{cell} is measured in a condition where there is no leak. For example, the actual pressure difference ΔP_{cell} is measured in a state in which none of the processes described above by way of example provides an indication of a leak.

If there is no leak, the gas sample Gp flows with a known volume flow rate Q_{ges} from the branch point 34 to the pressure sensor 57. Applying the formula (6) provides the calculation rule for the expected pressure difference ΔPₑₓₚ

$$Δ {P}_{e xp} = \{ {R}_{S g . 1} ( {Q}_{g e s} ) + {R}_{S g . 2} ( {Q}_{g e s} ) + {R}_{S g . 3} ( {Q}_{g e s} ) \} * {Q}_{g e s} . \qquad ( 17 )$$

If the measured actual pressure difference ΔP_{cell} deviates from the expected pressure difference ΔPₑₓₚ by more than a specified absolute or percentage threshold, at least one of the values used so far for the pneumatic resistances R_{Sg.1}(Q), R_{Sg.2}(Q), R_{Sg.3}(Q) deviates significantly from reality. These values must therefore be adapted to the changed reality.

In one embodiment, a corresponding message is output in a form that can be perceived by a person. A user can now empirically determine current values for the pneumatic resistances and cause these current values to be used.

In another embodiment, the values for the pneumatic resistors R_{Sg.1}(Q), R_{Sg.2}(Q), R_{Sg.3}(Q) are automatically adjusted. For example, a predetermined adjustment rule is applied to the previously used values for the pneumatic resistances R_{Sg.1}(Q), R_{Sg.2}(Q), R_{Sg.3}(Q), whereby this adjustment rule depends on the expected pressure difference ΔPₑₓₚ and the measured actual pressure difference ΔP_{cell}. For example, each value is changed by the same factor, whereby the factor is equal to the quotient ΔP_{cell}/ΔPₑₓₚ.

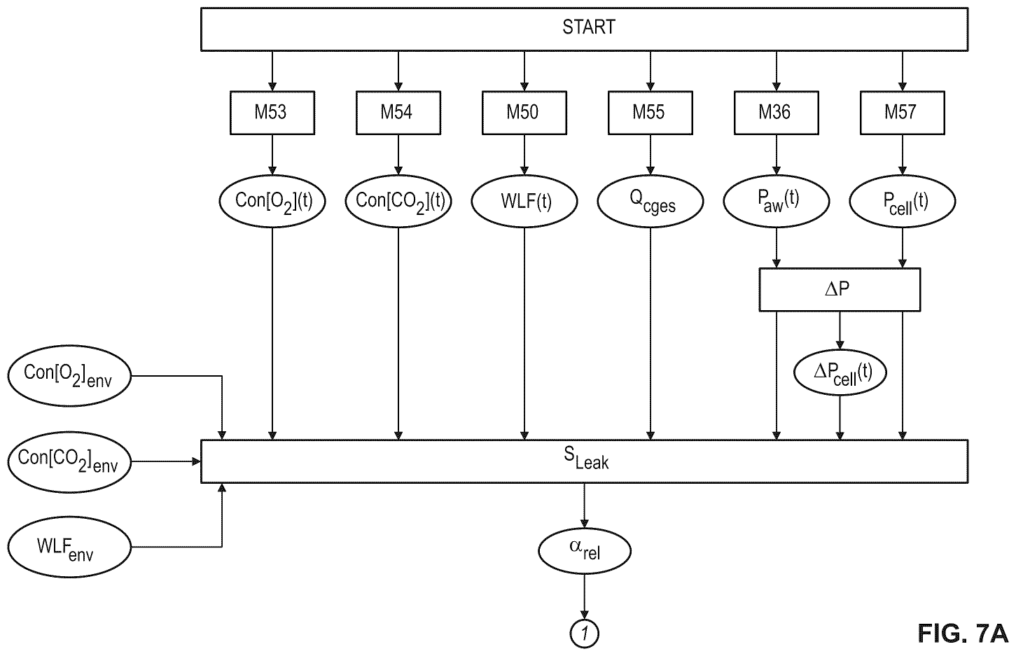

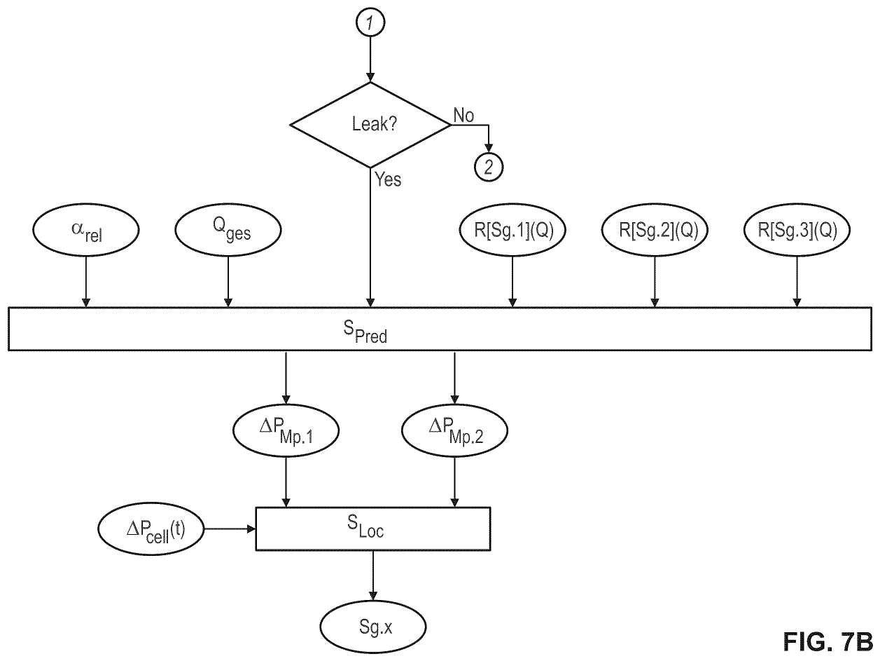

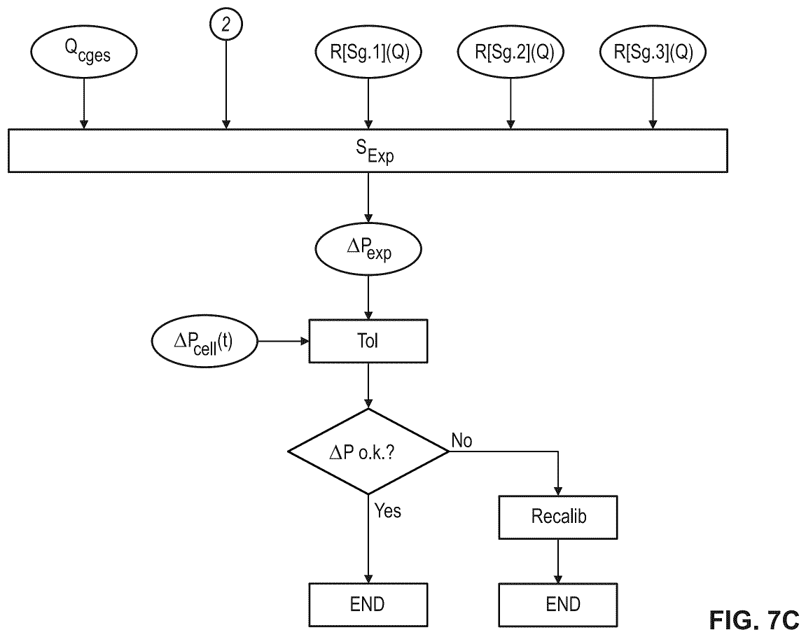

FIGS. 7A, 7B and 7C show the process steps of the embodiment example using a flow chart. FIGS. 7A, 7B and 7C indicate the following:
M53 The sensor 53 measures the time course Con[O₂](t) of the O₂ concentration in the measuring gas mixture MGg.
M54 The sensor 54 measures the time course Con[CO₂](t) of the CO₂ concentration in the measuring gas mixture MGg.
M50 The sensor 50 measures the time course WLF(t) of the thermal conductivity of the measuring gas mixture MGg.
M55 The total volume flow rate Q_{ges} to pump 55 is measured or captured.
M36 The pressure sensor 36 measures the pressure P_{aw}(t) in the ventilation circuit 40.
M57 The pressure sensor 57 measures the pressure P_{cell} (t) at the measuring position Mp.3 in the sensor arrangement 50.
ΔP The difference ΔP_{cell} (t) between the measured pressures P_{cell} (t) and P_{aw}(t) is calculated.
S_{Leak} It is checked whether a leak has occurred in the measuring system 100.
Leak? Decision: Leak in measuring system 100?
S_{Pred} The predicted pressure differences ΔP_{Mp.1} and ΔP_{Mp.2} are calculated.
S_{Loc} The segment Sg.x, in which the leak L has occurred, is identified (located/localized).
S_{Exp} The expected pressure difference ΔP exp in a leak-free state is calculated depending on the currently used pneumatic resistances of the segments.
Tol The deviation between the measured pressure difference ΔP_{cell} and the expected pressure difference ΔP exp is calculated.
ΔP o.k.? Decision: Is the calculated deviation ΔP_{cell} and ΔPₑₓₚ within a specified tolerance band?
Recalib An alarm is generated: The previously used value for the pneumatic resistance R_{Sg.1}(Q), R_{Sg.2}(Q), R_{Sg.3}(Q) of at least one segment no longer applies and a new value must be derived (recalibration required).

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Ventilator in the form of an anesthesia device, performs ventilation |
| | strokes, expels a quantity of the breathable gas mixture Gg in each |
| | ventilation stroke |
| 17 | Carrier gas line, leads from the mixer 48 to the anesthetic vaporizer |
| | 31, conducts the fluid flow 27 |
| 18 | Feed line, leads from the anesthetic vaporizer 31 to the feed point |
| | 38, conducts the fluid flow 28 |
| 20 | Line between the patient-side coupling unit 21 and the Y-piece 22 |
| 21 | Patient-side coupling unit, connected to the patient's body Pt and to |
| | the Y-piece 22 |
| 22 | Y-piece, connects the two gas lines 32 and 33 to the patient-side |
| | coupling unit 21 via line 20 |
| 23a | Non-return valve in the inspiratory gas line 32 |
| 23b | Non-return valve in the expiratory gas line 33 |
| 24a | Fluid conveying unit that moves the gas mixture Gg in the |
| | ventilation circuit 40 |
| 24b | Controllable proportional valve, causes the ventilation strokes |
| 24c | PEEP valve, maintains end-expiratory pressure in the lungs |
| 25a | Carbon dioxide absorber, extracts at carbon dioxide from the |
| | exhaled air |
| 26 | Line between the Y-piece 22 and the patient-side coupling unit 21 |
| 27 | Fluid flow with a carrier gas, flows in the carrier gas line 17 from |
| | the mixer 48 to the anesthetic vaporizer 31 |
| 28 | Fluid stream with a carrier gas and an anesthetic, flows in the |
| | supply line 18 from the anesthetic vaporizer 31 to the feed point 38 |
| 29 | Pressure relief valve in the ventilation circuit 40 |
| 30 | Signal processing unit of the sensor arrangement 50 |
| 31 | Anesthetic vaporizer, generates the fluid flow 28 with vaporous |
| | anesthetic from the fluid flow 27 with a carrier gas and from liquid |
| | anesthetic and feeds this into the feed line 18, comprises the |
| | vaporizer chamber 47 |
| 32 | Gas line for inhalation (inspiration), leads from the carbon dioxide |
| | absorber 25a to the Y-piece 22 |
| 33 | Gas line for exhalation (expiration), leads from the Y-piece 22 to |
| | the carbon dioxide absorber 25a |
| 34 | Branch-off point at which the gas sample Gp is branched off from |
| | the ventilation circuit 40 and fed into the sampling hose 52 |
| 35 | Ventilation control unit, controls the mixer 48 and the anesthetic |
| | vaporizer 31, processes signals from the sensors 36, 46, 39, 58 and |
| | from the sensor arrangement 50 |
| 36 | Pressure sensor that measures the pressure Paw in the ventilation |
| | circuit 40 |
| 37 | Entry point at which the gas sample Gp is fed back into the |
| | ventilation circuit 40 |
| 38 | Feed point at which the fluid flow 28 is fed into the ventilation |
| | circuit 40 |
| 39 | Temperature sensor, measures the ambient temperature |
| 40 | Ventilation circuit between the anesthesia apparatus 1 and the |
| | patient-side coupling unit 21, in which a gas mixture Gg is |
| | conveyed to the patient-side coupling unit 21 and exhaled air At is |
| 44 | conveyed back to the anesthesia apparatus 1 |
| | Conditioner, extracts breath containing anesthetics from exhaled |
| | breath At, |
| 46 | Volume flow rate sensor, measures the volume flow rate Vol' |
| | through the pipe 32 |
| 47 | Vaporization chamber of the anesthetic vaporizer 31 |
| 48 | Mixer, generates the carrier gas from the components breathing air |
| | and / or pure oxygen (O₂) and optionally nitrous oxide (N₂O) and |
| | feeds the carrier gas into the carrier gas line 17 |
| 50 | Sensor arrangement, analyzes the measuring gas mixture MGg, |
| | comprises the sensors 53, 54, 57 and 59, the pump 55, the fluid |
| | guide unit 60 and the signal processing unit 30 |
| 51 | Water trap at the input of the sensor arrangement 50 |
| 52 | Sampling hose for the gas sample Gp, starts at branch point 34 and |
| | leads to the sensor arrangement 50 |
| 53 | Sensor for the oxygen concentration in the measuring gas mixture |
| | MGg |
| 54 | Sensor for the carbon dioxide concentration in the measuring gas |
| | mixture MGg |
| 55 | Pump of the sensor arrangement 50, draws the gas sample Gp from |
| | the ventilation circuit 40 |
| 56 | Return hose for the gas sample Gp, starts in the sensor arrangement |
| | 50 and leads to the entry point 37 |
| 57 | Pressure sensor that measures the pressure P_{cell} of the measuring gas |
| | mixture MGg at the measuring position Mp.3 in the sensor |
| | arrangement 50 |
| 58 | Pressure sensor that measures the air pressure P_{amb} in the |
| | environment |
| 59 | Sensor for the anesthetic concentration in the measuring gas mixture |
| | MGg |
| 60 | Fluid guide unit through the sensor arrangement 50 |
| 100 | Measuring system, comprises the sensor arrangement 50, the water |
| | trap 51, the signal processing unit 30 and the hoses 52 and 56 |
| 200 | Ventilation arrangement, comprising the ventilator 1, the lines 32 |
| | and 33, the Y-piece 22, the patient-side coupling unit 21, the |
| | measuring system 100, the sensors 36 and 46 and the optional |
| | sensors 39 and 58, provides the ventilation circuit 40 |
| αᵣₑₗ | Relative leakage volume flow rate, proportion of the volume flow |
| | rate Q_{Leak} through the leak L in the total volume flow rate Q_{ges} |
| At | Gas mixture which is exhaled by the patient Pt into the patient-side |
| | coupling unit 21 and flows in the ventilation circuit 40 to the |
| | anesthesia device 1 |
| Con[CO₂]ₑₙᵥ | Proportion of CO₂ in the ambient air, is given or measured in |
| | advance |
| Con[CO₂](t) | Time course of the proportion of CO₂ in the measuring gas mixture |
| | MGg, measured by sensor 54 |
| Con[O₂]ₑₙᵥ | Proportion of O₂ in the ambient air, is given or measured in advance |
| Con[O₂](t) | Time course of the proportion of O₂ in the measuring gas mixture |
| | MGg, is measured by sensor 53 |
| Gg | Breathable gas mixture, which is expelled from the anesthesia |
| | device 1 and fed through the line 32 to the patient-side coupling unit |
| | 21, contains an anesthetic and a carrier gas with oxygen |
| Gp | Gas sample, which is branched off from the ventilation circuit 40 at |
| | branch point 34 and fed back into the ventilation circuit 40 at entry |
| | point 37 |
| L | Leak in the measuring system 100 |
| MGg | Measuring gas mixture flowing through the sensor arrangement 50 |
| | is composed of the gas sample Gp and a gas mixture flowing |
| | through the leak L, is equal to the gas sample Gp in a leak-free state |
| Mp.0 | Measuring position at branch point 34 |
| Mp.1 | Measuring position at the transition between the two segments Sg.1 |
| | and Sg.2 |
| Mp.3 | Measuring position at the transition between the two segments Sg.2 |
| | and Sg.3 |
| Mp.3 | Measuring position of pressure sensor 57, also the downstream end |
| | of segment Sg.3 |
| Pt | Patient, is connected to the patient-side coupling unit 21, is |
| | artificially ventilated and optionally anesthetized |
| P_{amb} | Ambient pressure, measured by pressure sensor 58 |
| P_{aw} | Airway pressure at the patient-side coupling unit 21, measured by |
| | the pressure sensor 36 on the line 20 |
| P_{cell} | Time-varying pressure at measuring position Mp.3, measured by |
| | pressure sensor 57 |
| ΔP_{cell} | Time-varying pressure loss between the pressure P_{aw} at the branch |
| | point 34 and the pressure P_{cell} in the sensor arrangement 50 |
| ΔPₑₓₚ | Expected pressure difference in a leak-free state is predicted using |
| | the current values for the pneumatic resistances R_{Sg.1}(Q), R_{Sg.2}(Q), |
| | R_{Sg.3}(Q) |
| ΔP_{Gp} | Pressure difference that the gas sample Gp experiences on the way |
| | from measuring position Mp.0 to measuring position Mp.3 |
| ΔP_{Mp.1} | Predicted difference between the pressures P_{cell} and P_{aw} if the leak L |
| | occurs at the measuring position Mp.1 |
| ΔP_{Mp.2} | Predicted difference between the pressures P_{cell} and P_{aw} if the leak L |
| | occurs at the measuring position Mp.2 |
| R | Pneumatic resistance |
| R_{Sg.1}(Q), | Pneumatic resistance of the segment Sg.1, Sg.2, Sg.3, depends on |
| R_{Sg.2}(Q), | the volume flow rate Q through the segment |
| R_{Sg.3}(Q) | |
| Q | Volume flow rate |
| Q_{ges} | Total volume flow rate in [l/min] of the measuring gas mixture |
| | MGg flowing to the pressure sensor 57, is generated and measured |
| | or derived from the pump 55 or from a positive pressure in the |
| | ventilation circuit 40 |
| Q_{Leak} | Volume flow rate through the leak L |
| Q_{Sg.1}, | Actual volume flow rate through the segment Sg.1, Sg.2, Sg.3 |
| Q_{Sg.2}, | |
| Q_{Sg.3} | |
| Sg.1 | Segment with the sampling hose 52, extends between the measuring |
| | positions Mp.0 and Mp.1 |
| Sg.2 | Segment with the water trap 51, extends between the measuring |
| | positions Mp.1 and Mp.2 |
| Sg.3 | Segment in the sensor arrangement 50, extends between the |
| | measuring positions Mp.2 and Mp.3 |
| T_{Leak} | Time period in which a leak L occurred |
| Temp_{amb} | Ambient temperature, measured by temperature sensor 39 |
| Vol′ | Volume flow rate (volume flow) through the line 32, measured by |
| | the volume flow rate sensor 46 |
| WLF(t) | Thermal conductivity of the measuring gas mixture MGg over time, |
| | measured by sensor 50 |
| WLFₑₙᵥ | Thermal conductivity of the ambient air, is given or measured in |
| | advance |

What is claimed is:

1. A monitoring process for monitoring a measuring system for medical treatment of a patient, the measuring system comprising: a sensor arrangement, which is configured to measure at least one property of a measuring gas mixture, which measuring gas mixture contains a gas sample to be analyzed; a pressure sensor arrangement; and a sensor fluid guide unit, wherein the monitoring process is carried out while via a patient fluid guide unit a fluid connection is at least temporarily established between a patient-side coupling unit, which coupling unit is arranged in and/or on the body of the patient, and a medical device,
the monitoring process comprising the steps of:
branching the gas sample off from the patient fluid guide unit at a branch point and guiding the gas sample through the sensor fluid guide unit and the sensor arrangement;
measuring a pressure at the branch point;
measuring a pressure in the measuring system at a measuring position in the sensor fluid guide unit or in the sensor arrangement; and
determining whether an indication of a leak has occurred in the sensor fluid guide unit;
wherein a section of the sensor fluid guide unit is divided into a segment sequence with at least two segments connected in series,
wherein the section leads from the branch point to the measuring position,
wherein for each segment a respective pneumatic resistance is given, which pneumatic resistance depends on a volume flow rate through the segment; and
upon determining that an indication of a leak has occurred, the monitoring process comprises the further steps of:
measuring or capturing a total volume flow rate of the measuring gas mixture through the sensor arrangement;
determining an actual pressure difference between the pressure measured at the measuring position and the pressure measured at the branch point;
determining an indicator for a relative leakage volume flow rate, the relative leakage volume flow rate being a ratio between a volume flow rate through a leak to be detected and the measured or captured total volume flow rate;
for each transition between a segment of the segment sequence and the next segment arranged downstream in the segment sequence, calculating a respective transition pressure difference,
wherein the transition pressure difference is a pressure difference caused when the leak occurs at the transition and
wherein the or each transition pressure difference is calculated based on the measured or captured total volume flow rate, the relative leakage volume flow rate, and the given pneumatic resistances of the segments; and
based on the determined actual pressure difference and the or each calculated transition pressure difference, identifying a segment which has caused the indication of the leak.

2. A monitoring process according to claim 1,
wherein the measuring system further comprises a fluid conveying unit, which fluid conveying unit can be switched on and off,
wherein the switched-on fluid conveying unit is configured to branch off the gas sample to be examined from the patient fluid guide unit and to convey the gas sample to the sensor arrangement, and
wherein the step of measuring the pressure in the measuring system is carried out while the fluid conveying unit is switched-off.

3. A monitoring process according to claim 1,
wherein the step of determining the indicator for the relative leakage volume flow rate comprises the step of measuring, with the sensor arrangement, a proportion of a component of the measuring gas mixture, as the or one property of a measuring gas mixture, and
wherein the indicator for the relative leakage volume flow rate is determined based on a measured proportion of the component of the measuring gas mixture, the total volume flow rate, and a given proportion of the component in a gas mixture in the environment of the measuring system, and/or
wherein the step of determining the indicator for the relative leakage volume flow rate comprises the step of measuring, with the sensor arrangement, a thermal conductivity of the measuring gas mixture as the or one property of a measuring gas mixture, and
wherein the indicator for the relative leakage volume flow rate is determined based on the measured thermal conductivity of the measuring gas mixture, the total volume flow rate, and a given thermal conductivity of a gas mixture in the environment of the measuring system.

4. A monitoring process according to claim 1,
wherein the determination as to whether an indication of a leak has occurred is based on the determined relative leakage volume flow rate.

5. A monitoring process according to claim 1,
wherein the sensor arrangement measures a respective time course of a proportion of at least two different constituents of the measuring gas mixture, and
wherein the determination as to whether an indication of a leak has occurred is made depending on the two measured time courses.

6. A monitoring process according to claim 1,
wherein upon determining that no indication of a leak has occurred the monitoring process comprises the step of calculating an expected pressure difference between the pressure at the measuring position and the pressure at the branch point,
wherein for the calculation of the expected pressure difference the measured or captured total volume flow rate and the given pneumatic resistances of the segments are used, and if, as a result, the determined actual pressure difference deviates from the calculated expected pressure difference by more than a given difference threshold, a message comprising this result is generated.

7. A monitoring process according to claim 6,
wherein the generated result is output in a form that can be perceived by a human.

8. A monitoring process according to claim 1,
wherein the medical treatment comprises artificial ventilation of the patient.

9. A monitoring process according to claim 1,
wherein the at least one property of the measuring gas mixture is a proportion of a component of the measuring gas mixture.

10. A measuring process for medical treatment, wherein the measuring process is carried out using a measuring system, the measuring system comprising: a sensor arrangement; a pressure sensor arrangement; and a sensor fluid guide unit,
wherein the measuring process is carried out while a patient fluid guide unit establishes at least temporarily a fluid connection between a patient-side coupling unit, which coupling unit is arranged in and/or on the body of a patient, and a medical device,
wherein the measuring process comprises the steps of:
branching off a gas sample from the patient fluid guide unit at a branch point and guiding the gas sample through the sensor fluid guide unit and the sensor arrangement;
with the sensor arrangement, measuring at least one property of a measuring gas mixture, which measuring gas mixture contains the branched-off gas sample,
measuring a pressure at the branch point;
measuring a pressure in the measuring system at a measuring position in the sensor fluid guide unit or in the sensor arrangement; and
determining whether an indication of a leak has occurred in the sensor fluid guide unit;
wherein a section of the sensor fluid guide unit is divided into a segment sequence with at least two segments connected in series,
wherein the section leads from the branch point to the measuring position, and
wherein for each segment a respective pneumatic resistance is given, which pneumatic resistance depends on a volume flow rate through the segment; and
upon determining that an indication of a leak has occurred, the measuring process comprises the further steps of:
measuring or capturing a total volume flow rate at which the measuring gas mixture flows through the sensor arrangement;
determining an actual pressure difference between the pressure measured at the measuring position and the pressure measured at the branch point;
determining an indicator for a relative leakage volume flow rate, the relative leakage volume flow rate being a ratio between a volume flow rate through a leak to be detected and the measured or captured total volume flow rate;
for each transition between a segment of the segment sequence and the next segment arranged downstream in the segment sequence, calculating a respective transition pressure difference,
wherein the transition pressure difference is a pressure difference that is caused when the leak occurs at the transition and
wherein the or each transition pressure difference is calculated using the total volume flow rate, the relative leakage volume flow rate, and the given pneumatic resistances of the segments,
and
based on the determined actual pressure difference and the or each calculated transition pressure difference, identifying a segment which has caused the indication of the leak.

11. A measurement process according to claim 10,
wherein the medical treatment comprises artificial ventilation of the patient.

12. A measurement process according to claim 10,
wherein the at least one property of the measuring gas mixture is a proportion of a component of the measuring gas mixture.

13. A monitoring device for monitoring a measuring system for medical treatment of a patient,
wherein the measuring system comprises: a sensor arrangement, which sensor arrangement is configured to measure at least one property of a measuring gas mixture, wherein the measuring gas mixture contains a gas sample to be analyzed; a pressure sensor arrangement; and a sensor fluid guide unit,
wherein the measuring system is configured to be at least temporarily connected to a patient fluid guide unit, which patient fluid guide unit is configured to establish a fluid connection between a patient-side coupling unit, which coupling unit is arranged in and/or on the body of the patient, and a medical device,
wherein the measuring system is configured to branch off the gas sample from the patient fluid guide unit at a branch point and guide the gas sample through the sensor fluid guide unit and the sensor arrangement,
wherein the pressure sensor arrangement is configured to measure a pressure at the branch point and to measure a pressure at a measuring position in the sensor fluid guide unit or in the sensor arrangement,
wherein a section of the sensor fluid guide unit is subdivided into a segment sequence comprising at least two segments connected in series,
wherein the section leads from the branch point to the measuring position and
wherein for each segment a respective pneumatic resistance is given, which pneumatic resistance depends on a volume flow rate through the segment,
wherein the monitoring device is configured to determine whether an indication of a leak has occurred in the sensor fluid guide unit,
wherein the monitoring device is configured, upon determining an indication of a leak, to:
measure or capture a total volume flow rate at which the measuring gas mixture flows through the sensor arrangement;
determine the actual pressure difference between the pressure measured at the measuring position and the pressure measured at the branch point;
determine an indicator for a relative leakage volume flow rate, wherein the relative leakage volume flow rate is a ratio between the volume flow rate through a leak to be detected and the measured or captured total volume flow rate;
calculate for each transition between a segment of the segment sequence and the next segment arranged downstream in the segment sequence a respective transition pressure difference, wherein the transition pressure difference is a pressure difference that is caused when the leak occurs at the transition; and
based on the determined actual pressure difference and the or each calculated transition pressure difference, identify a segment that has caused the indication of the leak;
wherein the monitoring device is configured to calculate the or each transition pressure difference using the total volume flow rate, the relative leakage volume flow rate, and the given pneumatic resistances of the segments.

14. A monitoring device according to claim 13,
wherein the medical treatment comprises artificial ventilation of the patient.

15. A monitoring device according to claim 10,
wherein the at least one property of the measuring gas mixture is a proportion of a component of the measuring gas mixture.

16. A measuring system for medical treatment of a patient, wherein the measuring system comprises:
a sensor arrangement, which is configured to measure at least one property of a measuring gas mixture, which measuring gas mixture contains a gas sample to be analyzed;
a pressure sensor arrangement;
a sensor fluid guide unit; and
a monitoring device,
wherein the measuring system is configured to be connected to a patient fluid guide unit, which patient fluid guide unit is configured to establish at least temporarily a fluid connection between a patient-side coupling unit, which coupling unit is arranged in and/or on the body of the patient, and a medical device,
wherein the measuring system is configured to branch off the gas sample from the patient fluid guide unit at a branch point and to guide the gas sample through the sensor fluid guide unit to the sensor arrangement,
wherein the pressure sensor arrangement is configured to measure a pressure at the branch point and a pressure at a measuring position in the sensor fluid guide unit or in the sensor arrangement,
wherein a section of the sensor fluid guide unit, which section leads from the branch point to the measuring position, is subdivided into a segment sequence comprising at least two segments connected in series,
wherein for each segment a respective pneumatic resistance is given, which pneumatic resistance depends on a volume flow rate through the segment,
wherein the monitoring device is configured to determine whether an indication of a leak has occurred in the sensor fluid guide unit;
wherein the monitoring device is configured, upon determining an indication of a leak, to:
measure or capture a total volume flow rate at which the measuring gas mixture flows through the sensor arrangement;
determine the actual pressure difference between the pressure measured at the measuring position and the pressure measured at the branch point;
determine an indicator for a relative leakage volume flow rate, wherein the relative leakage volume flow rate is a ratio between the volume flow rate through a leak to be detected and the measured or detected total volume flow rate;
for each transition between a segment of the segment sequence and the next segment arranged downstream in the segment sequence, calculate a respective transition pressure difference,
wherein the transition pressure difference is a pressure difference that is caused when the leak occurs at the transition; and
based on the determined actual pressure difference and the or each calculated transition pressure difference, identify a segment that has caused the indication of the leak;
wherein the monitoring device is configured to calculate the or each transition pressure difference using the total volume flow rate, the relative leakage volume flow rate, and the given pneumatic resistances of the segments.

17. A measuring system according to claim 14,
wherein the medical treatment comprises artificial ventilation of the patient.

18. A measuring system according to claim 14,
wherein the at least one property of the measuring gas mixture is a proportion of a component of the measuring gas mixture.

19. A measuring system for monitoring a medical treatment of a patient according to claim 14, in combination with: the medical device; the patient fluid guide unit; and the patient-side coupling unit to form a medical treatment arrangement for medical treatment.

20. A measuring system according to claim 19,
wherein the at least one property of the measuring gas mixture is a proportion of a component of the measuring gas mixture.
